(19) United States
(12) Patent Application Publication
(10) Pub. No.: US 2019/0231847 A1
(43) Pub. Date: Aug. 1, 2019

(54) TREATMENT OF OCULAR DISEASE

(71) Applicant: ALMAC DISCOVERY LIMITED, Craigavon (GB)

(72) Inventor: Tara MOORE, Craigavon (GB)

(73) Assignee: ALMAC DISCOVERY LIMITED, Craigavon (GB)

(21) Appl. No.: 16/312,234

(22) PCT Filed: Jun. 21, 2017
(86) PCT No.: PCT/GB2017/051820

§ 371 Date: Dec. 20, 2018

(30) Foreign Application Priority Data
Jun. 22, 2016 (GB) 1610938.1
Apr. 4, 2017 (GB) 1705435.4

(51) Int. Cl. A61K 38/17; A61P 27/02; A61K 9/00

(52) U.S. Cl. CPC A61K 38/17; A61K 9/0051; A61K 9/0019; A61P 27/02

(57) ABSTRACT

A treatment of ocular disease, and more specifically disorders of the cornea, using the polypeptide FKBP-L and peptide fragments thereof is provided.

### FIELD OF THE INVENTION

The invention relates to treatment of ocular disease, and more specifically disorders of the cornea, using the polypeptide FKBP-L and peptide fragments thereof.

### BACKGROUND OF THE INVENTION

A healthy cornea is essential for vision. According to the WHO, corneal blindness is the fourth leading cause of blindness globally, after cataract, glaucoma and age-related macular degeneration. Worldwide, more than 7 million people are blinded annually due to corneal scarring and the abnormal growth of blood vessels (neovascularization) in the eye. In clinical situations where corneal damage occurs it is imperative to minimize scar formation and hence minimise blindness. The current treatment for neovascularization-induced corneal opacity damage is corneal graft, however, the presence of neovascularization itself contributes to a worse prognosis following grafting in this high-risk group. When corneal grafts are placed into an avascular recipient bed (low-risk keratoplasty), 2-year graft survival rates approach 90% under cover of topical steroids (Küchle et al., 2002). In vascularized, recipient beds (high-risk keratoplasty) the graft survival for 2 years decreases significantly below 50% (Cursiefen et al., 2002). Sutures invariably induce a significant neovascular response, which is also associated with an increased risk of corneal rejection; therefore even the means by which a corneal graft is fastened in place by itself increases the risk of graft rejection. New therapeutic options, such as those presented in this application, are desperately needed, as currently available treatments for this type of eye disease, such as corticosteroids, have limited efficacy and a risk of side effects.

Dry eye syndrome is an extremely common problem affecting up to 30% of the population (Clegg et al., 2006), in severe cases it can result in damage to the ocular surface associated with significant inflammation. Inflammation in dry eye syndrome is associated with infiltration of inflammatory cells and upregulated expression of immune markers. The condition is principally managed by corticosteroids and/or the T cell inhibitor cyclosporine (Pflugfelder, 2003). The known associated complications of long-term corticosteroids make a search for an alternative efficacious treatment a high priority. The lack of tolerance to cyclosporin due to stinging and discomfort and its lack of efficacy in a significant number of patients mean the quest for other efficacious treatments is extremely important.

Childhood ocular rosacea otherwise called blepharokeratoconjunctivitis is an uncommon condition, which is characterized by chronic posterior and anterior blepharitis. In some cases associated ocular inflammation can be significant resulting in corneal infiltrates, ulceration and corneal vascularisation (Doan et al., 2007). The underlying pathology is thought to be due to a primary meibomitis, followed by bacterial overgrowth and T cell mediated inflammation. The mainstay of treatment is a combination of oral or topical antibiotics, topical steroids, and/or cyclosporin as a steroid reducing agent. Corneal vascularisation, which can occur with this condition often, occurs quickly and aggressively, requiring high does topical steroid to reduce or halt its progression. This condition requires better management strategies, which are effective and reduce the current requirement for long-term use of topical steroids on children's eyes.

Most treatment strategies to induce regression of blood vessels within the cornea have utilized anti VEGF treatments, this type of treatment is often quite successful if it occurs early in the condition, however once blood vessels are well established they become unresponsive to anti VEGF treatments. The difficulties with any of the current anti VEGF approaches are that they often require multiple injections over a long period, particularly as the condition often waxes and wanes producing more inflammation and promoting further vascularisation. Currently no medical treatment is available to remove blood vessels, which are well established within the cornea. The only treatments in these circumstances are through the use of either laser treatments or cautery, both of which are far from satisfactory often requiring multiple surgical interventions (Gupta & Illingworth, 2011).

There is a clinical need to provide new therapeutics that can reduce or prevent corneal neovascularisation and/or reduce or prevent inflammatory eye disease. Such therapeutics may be important as stand-alone treatments, or to be used in conjunction with other therapeutic agents.

### SUMMARY OF THE INVENTION

FKBP-L polypeptide and peptide fragments thereof have previously been described as anti-angiogenic agents with clinical potential in the treatment of cancer, specifically solid tumours.

Peptide fragments of FKBP-L have now been tested in animal models of ocular disease, e.g. the rat corneal suture model and the mouse experimental autoimmune uveoretinitis (EAU) model. Surprisingly, it has been observed that topical administration of FKBP-L peptide fragments to the cornea results in a substantial reduction in blood vessel formation. In addition, the FKBP-L peptide also acts as an anti-inflammatory agent in the eye, both when injected into the eye and when administered parenterally (intraperitoneally). These experimental findings support the clinical utility of FKBP-L, and biologically active peptide fragments thereof, in the treatment of a number of ocular diseases characterised by/associated with neovascularisation and/or inflammation.

Therefore, in accordance with a first aspect of the invention there is provided FKBP-L polypeptide or a biologically active peptide fragment thereof for use in the treatment or prevention of corneal neovascularisation.

In one embodiment there is provided FKBP-L polypeptide or a biologically active peptide fragment thereof for use in the prevention or treatment of corneal neovascularisation following corneal graft surgery

In a further embodiment there is provided FKBP-L polypeptide or a biologically active peptide fragment thereof for use in the treatment or prevention of an inflammatory disorder of the eye.

In certain embodiments, the inflammatory disorder of the eye is uveitis, dry eye syndrome or blepharokeratoconjunctivitis.

The invention further provides a method of treating or preventing corneal neovascularisation in a mammalian subject, comprising administering to a subject in need thereof a therapeutically effective amount of an FKBP-L polypeptide or a biologically active peptide fragment thereof.

One embodiment relates to a method of treating or preventing corneal neovascularisation in a subject who has undergone corneal graft surgery.

The invention still further provides a method of treating or preventing an inflammatory disorder of the eye in a mammalian subject, comprising administering to a subject in need thereof a therapeutically effective amount of an FKBP-L polypeptide or a biologically active peptide fragment thereof.

In certain embodiments, the inflammatory disorder of the eye is uveitis, dry eye syndrome or blepharokeratoconjunctivitis.

In one embodiment, the FKBP-L polypeptide or a biologically active peptide fragment thereof is to be administered topically to the eye.

In one embodiment, the FKBP-L polypeptide or a biologically active peptide fragment thereof is administered by subconjunctival injection, intrastromal injection or intraocular injection.

In preferred embodiments of each of the above-described aspects of the invention, the biologically active peptide fragment of FKBP-L used in said treatment/prevention comprises the amino acid sequence IRQQPRDPPTETLELEVSPDPAS (SEQ ID NO:3), or a sequence at least 90% identical thereto.

In further embodiments, the FKBP-L polypeptide used in said treatment/prevention comprises the amino acid sequence shown as SEQ ID NO:1 or SEQ ID NO:2, or a sequence at least 90% identical thereto.

In further embodiments, the biologically active peptide fragment of FKBP-L used in said treatment/prevention comprises the amino acid sequence shown as any one of SEQ ID Nos 4 to 23, or a sequence at least 90% identical thereto.

In preferred embodiments, the subject to be treated is a human.

Features of the invention will be described in further detail hereinafter. It is to be understood that the invention is not limited in its application to the details set forth in the following claims, description and figures. The invention is capable of other embodiments and of being practiced or carried out in various ways.

### BRIEF DESCRIPTION OF THE DRAWINGS

The invention will be further understood with reference to the following drawings.

FIG. 1 Suture induced neovascularisation in rats. Black arrows indicate position of sutures and white arrow indicates neovascularisation.

FIG. 2 Subconjunctival injection—(A) Neovascularisation induced by sutures in triamcinolone treated rats (left panel), PBS control rats (centre panel) and ALM201 treated rats (right panel). (B) Effect of subconjunctival injection of Triamcinolone or ALM201 compared to control.

FIG. 3 Topical treatment—(A) Neovascularisation induced by sutures in PBS control rats (left panel) and ALM201 treated rats (right panel). (B) Effect of topical ALM201 compared to topical control.

FIG. 4 Suture-induced neovascularisation in Hooded Lister Rats treated with subconjunctival injection of dexamethasone (left panel), ALM201 (right panel), or PBS control (centre panel).

FIG. 5 Bodyweight of experimental mice during the course of treatment. Bodyweights were measured once daily during the 10-day treatment period. Arrows indicate the drop of bodyweight in two mice during day 3-5 in ALM201+Dexamethasone treatment group (E).

FIG. 6 Retinal inflammation and clinical score in EAU mice. (A) An example fundus image from inflamed mouse retina. (B-F) Mice were immunised with IRBP peptide 1-20. From day 14 p.i. mice were treated once daily with PBS (B), or ALM201 0.3 mg/kg (C), or AML201 3 mg/kg (D), Dexamethasone 0.5 mg/kg (E), or ALM201 0.3 mg/kg+Dexamethasone 0.5 mg/kg (F). Fundus images (B-E) were taken on day 24 p.i., and clinical scores (G) were obtained using a standard grading system (Xu et al. 2008a).

FIG. 7 Clinical score of EAU in different groups of mice showing changes over time in the EAU model with ALM201 treatment. Fundus images were taken from experimental mice at day 14 p.i. and day 24 p.i. using the TEFI system. Clinical disease was graded using a standard scoring system described previously (Xu et al. 2008a). **, P<0.01, paired Student's t test.

FIG. 8 Histology and histological scores of EAU in different groups of mice. After 10 days therapy, mice were sacrificed and eyes collected for standard H & E staining. At least three sections from different layers of each eye were used for histological score analysis. (A) image from a PBS treated EAU mouse. (B) image from a ALM201 0.3 mg/kg treated EAU mouse. (C-D) images from ALM201 3 mg/kg treated mice. (E) image from a dexamethasone treated mouse. (F, G) images from Dexamethasone+ALM201 3 mg/kg treated mice. (H) histological scores for each treatment group of mice was collated and plotted as a scatter plot. L, Lens; Vi, Vitreous cavity; GL, Ganglion Layer; INL, Inner Nuclear Layer; ONL, Outer Nuclear Layer; Ch, Choroid; POS, Photoreceptor Outer Segments. *, P<0.05; **, P<0.01; ***, P<0.001 compared to PBS treated group, Mann-Whitney U test.

FIG. 9 (A) FTICR Mass spectrum of peptide at m/z 2576.303±0.005 Da. Standard (STD) 100 nM spotting off tissue, a2) STD 100 nM on tissue, a3) theoretical monoisotopic distribution of peptide. Topically treated corneal tissue with a4) 100 μM and a5) 100 nM peptide. Observed peptide monoisotopic mass in agreement with the theoretical distribution (mass accuracy was <5 ppm at 350K mass resolution power); (B) MALDI-FTICR-MSI heat maps distribution of ALM201 at m/z 2576.303±0.005 Da; (C) Heat maps of: Endogenous metabolites at m/z 1028.135±0.025 Da mostly distributed in the cornea, 1444.584±0.025 Da mostly distributed in the lens, 782.5799±0.025 Da mostly distributed in the aqueous humour and vitreous humour, 780.5451±0.025 Da distributed in the muscle (blue), 835.5891±0.025 Da and the peptide distributed throughout the eye 2576.303±0.025 Da. Signal intensity is depicted on the scale shown. Scale bar is 1000 μm. Data was normalised by RMS. Spatial resolution is 40-50 μm.

FIG. 10 A superimposition of H&E stained rat eye section and MALDI-MSI image at 2576.3±0.1 Da. A normal eye was treated daily with 100 μM of ALM201 for 3 days and then enucleated 15 minutes after the last treatment. The image shows that the majority of the peptide co-localises with the vitreous humour and possibly the lens. The peptide is also co-localised in the cornea, sclera, choroid and retina (spatial resolution is −20 μm).

FIG. 11 Histological analysis by haematoxylin and eosin (H&E) staining showing increased infiltration of cells in control samples (PBS or dexamethasone) compared to treated samples (ALM201, 1 μM or 100 μM) at day 6 after suture and treatment. (A) Haematoxylin and eosin stained sections for each of the experimental groups. White arrows indicate sutures and black arrows show blood vessels (scale bar=500 μm). Lower panel shows distribution of CD68+ cells in sections from each treatment group. (B) The number of total infiltrated cells and (C) number of CD68+ infiltrated cells in each experimental group was quantitated, with higher numbers of infiltrated cells present in the control group compared to the peptide treated group.

FIG. 12 A schematic diagram of ALM201 concentration titration experiment described in Example 4

FIG. 13 (A) Clinical photographs of rat eyes after 6 days of treatment with PBS (vehicle control), dexamethasone (positive control) and several different concentrations of ALM201 (0.01 μM, 0.1 μM, 1 μM, 10 μM and 100 μM); (B-D) clinical scoring graphs showing vessel distance to suture (B), vessel density (C) and inflammation (D) in each concentration of ALM201 along with controls PBS and dexamethasone.

FIGS. 14 H&E images show changes in histology of the corneal epithelium and supporting stromal tissue following suture insertion and then following treatment with ALM201 peptide. This staining was used to count cell infiltrate and to find the sutured area. Black arrow=suture.

FIG. 15 Adjacent slides were used for immunohistochemistry staining to show the differences in CD44 expression in the indicated treatment groups.

FIG. 16 Adjacent slides were used for immunohistochemistry staining to show the differences in FKBPL expression in the indicated treatment groups.

FIG. 17 Immunohistochemistry staining using anti-NFκB p65 antibody and DAPI in the indicated treatment groups.

FIG. 18 Immunohistochemistry staining using anti-p-IκBα p65 antibody and DAPI in the indicated treatment groups.

### DETAILED DESCRIPTION OF THE INVENTION

### Definitions

Unless defined otherwise, all technical and scientific terms used herein have the same meaning as commonly understood by one of ordinary skill in the art. Practitioners are particularly directed to Current Protocols in Molecular Biology (Ausubel) for definitions and terms of the art. Abbreviations for amino acid residues are the standard 3-letter and/or 1-letter codes used in the art to refer to one of the 20 common L-amino acids.

Any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

Also, the terms “portion” and “fragment” are used interchangeably to refer to parts of a polypeptide, nucleic acid, or other molecular construct.

As used herein, the term “biologically active FKBP-L peptide” (e.g., fragment and/or modified polypeptides) is used to refer to a peptide or polypeptide that displays the same or similar amount and type of activity as the full-length FKBP-L polypeptide. In this context “biological activity” of an FKBP-L polypeptide, fragment or derivative includes any one of anti-angiogenic activity, inhibition of blood vessel formation and/or growth of blood vessels and anti-inflammatory activity. Biological activity of FKBP-L fragments or derivatives may be tested in comparison to full length FKBP-L using any of the in vitro or in vivo assays described in the accompanying examples, such as for example the rat corneal suture model or EAU model.

As used herein a “subject” may be an animal. For example, the subject may be a mammal. Also, the subject may be a human. In alternate embodiments, the subject may be either a male or a female. In certain embodiments, the subject may be a patient, where a patient is an individual who is under medical care and/or actively seeking medical care for a disorder or disease.

“Polypeptide” and “protein” are used interchangeably herein to describe protein molecules that may comprise either partial or full-length proteins. The term “peptide” is used to denote a less than full-length protein or a very short protein unless the context indicates otherwise.

As is known in the art, “proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. Typically, the amino acids making up a protein are numbered in order, starting at the amino terminal residue and increasing in the direction toward the carboxy terminal residue of the protein.

The terms “identity” or “percent identical” refers to sequence identity between two amino acid sequences or between two nucleic acid sequences. Percent identity can be determined by aligning two sequences and refers to the number of identical residues (i.e., amino acid or nucleotide) at positions shared by the compared sequences. Sequence alignment and comparison may be conducted using the algorithms standard in the art (e.g. Smith and Waterman, 1981, Adv. Appl. Math. 2:482; Needleman and Wunsch, 1970, J. Mol. Biol. 48:443; Pearson and Lipman, 1988, Proc. Natl. Acad. Sci., USA, 85:2444) or by computerized versions of these algorithms (Wisconsin Genetics Software Package Release 7.0, Genetics Computer Group, 575 Science Drive, Madison, Wis.) publicly available as BLAST and FASTA. Also, ENTREZ, available through the National Institutes of Health, Bethesda Md., may be used for sequence comparison. When utilizing BLAST and Gapped BLAST programs, the default parameters of the respective programs (e.g., BLASTN; available at the Internet site for the National Center for Biotechnology Information) may be used. In one embodiment, the percent identity of two sequences may be determined using GCG with a gap weight of 1, such that each amino acid gap is weighted as if it were a single amino acid mismatch between the two sequences. Or, the ALIGN program (version 2.0), which is part of the GCG (Accelrys, San Diego, Calif.) sequence alignment software package may be used.

As used herein, the term “conserved residues” refers to amino acids that are the same among a plurality of proteins having the same structure and/or function. A region of conserved residues may be important for protein structure or function. Thus, contiguous conserved residues as identified in a three-dimensional protein may be important for protein structure or function. To find conserved residues, or conserved regions of 3-D structure, a comparison of sequences for the same or similar proteins from different species, or of individuals of the same species, may be made.

As used herein, the term “similar” or “homologue” when referring to amino acid or nucleotide sequences means a polypeptide having a degree of homology or identity with the wild-type amino acid sequence. Homology comparisons can be conducted by eye, or more usually, with the aid of readily available sequence comparison programs. These commercially available computer programs can calculate percent homology between two or more sequences (e.g. Wilbur, W. J. and Lipman, D. J., 1983, Proc. Natl. Acad. Sci. USA, 80:726-730). For example, homologous sequences may be taken to include an amino acid sequences which in alternate embodiments are at least 70% identical, 75% identical, 80% identical, 85% identical, 90% identical, 95% identical, 96% identical, 97% identical, or 98% identical to each other.

As used herein, the term “at least 90% identical thereto” includes sequences that range from 90 to 99.99% identity to the indicated sequences and includes all ranges in between. Thus, the term at least 90% identical thereto includes sequences that are 91, 91.5, 92, 92.5, 93, 93.5. 94, 94.5, 95, 95.5, 96, 96.5, 97, 97.5, 98, 98.5, 99, 99.5 percent identical to the indicated sequence. Similarly the term “at least 70% identical includes sequences that range from 70 to 99.99% identical, with all ranges in between. The determination of percent identity is determined using the algorithms described herein.

As used herein, the term “linked” identifies a covalent linkage between two different groups (e.g., nucleic acid sequences, polypeptides, polypeptide domains) that may have an intervening atom or atoms between the two groups that are being linked. As used herein, “directly linked” identifies a covalent linkage between two different groups (e.g., nucleic acid sequences, polypeptides, polypeptide domains) that does not have any intervening atoms between the two groups that are being linked.

The term “peptide mimetics” refers to structures that serve as substitutes for peptides in interactions between molecules (Morgan et al., 1989, Ann. Reports Med. Chem., 24:243-252). Peptide mimetics may include synthetic structures that may or may not contain amino acids and/or peptide bonds but that retain the structural and functional features of a peptide, or agonist, or antagonist. Peptide mimetics also include peptoids, oligopeptoids (Simon et al., 1972, Proc. Natl. Acad, Sci., USA, 89:9367); and peptide libraries containing peptides of a designed length representing all possible sequences of amino acids corresponding to a peptide of the invention.

As used herein, an “effective amount” means the amount of an agent that is effective for producing a desired effect in a subject. The term “therapeutically effective amount” denotes that amount of a drug or pharmaceutical agent that will elicit therapeutic response of an animal or human that is being sought. The actual dose which comprises the effective amount may depend upon the route of administration, the size and health of the subject, the disorder being treated, and the like.

The term “pharmaceutical composition” is used herein to denote a composition that may be administered to a mammalian host, e.g. topically, systemically or intraocularly, in unit dosage formulations containing conventional non-toxic carriers, diluents, adjuvants, vehicles and the like.

The term “pharmaceutically acceptable carrier” as used herein may refer to compounds and compositions that are suitable for use in human or animal subjects, as for example, for therapeutic compositions administered for the treatment of a disorder or disease of interest.

A “stable” formulation is one in which the polypeptide or protein therein essentially retains its physical and chemical stability and biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation of interest may be kept at 40° C. for 1 week to 1 month, at which time stability is measured. The extent of aggregation following lyophilization and storage can be used as an indicator of peptide and/or protein stability. For example, a “stable” formulation is one wherein less than about 10% and preferably less than about 5% of the polypeptide or protein is present as an aggregate in the formulation. An increase in aggregate formation following lyophilization and storage of the lyophilized formulation can be determined. For example, a “stable” lyophilized formulation may be one wherein the increase in aggregate in the lyophilized formulation is less than about 5% or less than about 3%, when the lyophilized formulation is incubated at 40° C. for at least one week. Stability of the fusion protein formulation may be measured using a biological activity assay such as a binding assay as described herein.

### FKBP-L Polypeptides for the Treatment of Ocular Disease

The present invention is based on the observation that FKBP-L, and specifically peptide fragments of FKBP-L, can reduce the formation of corneal blood vessels and also exhibit anti-inflammatory activity when administered to the eye. These findings support the utility of FKBP-L, and peptide fragments thereof, in the treatment of a range of ocular disorders, and in particular ocular disorders associated with or mediated by corneal neovascularisation, and ocular disorders associated with or mediated by inflammation.

The term “FKBP-L” refers to the protein FK506 binding protein-like, (McKeen et al. Endocrinology, 2008, Vol 149(11), 5724-34; Gene ID:63943). FKBP-L and peptide fragments thereof have previously been demonstrated to possess potent anti-angiogenic activity (WO 2007/141533). The anti-angiogenic activity of FKBP-L peptide fragments appears to be dependent on an amino acid sequence located between amino acids 34-57, in the N-terminal region of the full-length protein. This anti-angiogenic activity suggested a clinical utility of the peptide in the treatment of cancers, particularly solid tumours. The present application extends beyonds the findings of WO 2007/141533 by demonstrating a specific clinical utility of FKBP-L and peptide fragments thereof in the treatment of ocular diseases, such as corneal neovascularisation and inflammatory disorders of the eye.

Embodiments of the present invention encompass the use of the full-length FKBP-L polypeptide, and also peptide fragments thereof which exhibit biological activity, as well as modified forms and derivatives of the full-length protein or biologically active peptide fragments, as therapeutic agents in the treatment of ocular disease.

The expression “FKBP-L polypeptide” is used in the specification according to its broadest meaning. It designates the naturally occurring full-length protein as shown in SEQ ID NO:1, together with homologues due to polymorphisms, other variants, mutants and portions of said polypeptide which retain their biological activities. For example, in certain embodiments, the FKBP-L polypeptide comprises SEQ ID NO:1 (GENBank Accession No. NP_071393; NM_022110; [gi:34304364]), or SEQ ID NO:2 with a Threonine at position 181 and a Glycine at position 186 of the wild-type sequence. Example constructs of other FKBP-L polypeptides (e.g., fragments and other modifications) and polynucleotide constructs encoding for FKBP-L polypeptides are described in WO 2007/141533, the contents of which are incorporated herein in their entirely by reference, expressly for this purpose.

In SEQ ID NO: 2, the FKBP-L insert (originally cloned into PUC18 by Cambridge Bioscience and now cloned into pcDNA3.1); had two inserted point mutations compared to the sequence that is deposited on the PUBMED database (SEQ ID NO: 1). There is a point mutation at 540 bp (from start codon): TCT to ACT which therefore converts a serine (S) to a Threonine (T) (amino acid: 181). There is also a point mutation at 555 bp (from start codon): AGG to GGG which therefore converts an Arginine (R) to a Glycine (G) (amino acid: 186). Both FKBP-L polypeptides (SEQ ID NO: 1 and SEQ ID NO: 2) display biological activity.

An FKBP-L polypeptide or peptide for use according to the present invention may include natural and/or chemically synthesized or artificial FKBP-L peptides, peptide mimetics, modified peptides (e.g., phosphopeptides, cyclic peptides, peptides containing D- and unnatural amino-acids, stapled peptides, peptides containing radiolabels), or peptides linked to antibodies, carbohydrates, monosaccharides, oligosaccharides, polysaccharides, glycolipids, heterocyclic compounds, nucleosides or nucleotides or parts thereof, and/or small organic or inorganic molecules (e.g., peptides modified with PEG or other stabilizing groups). Thus, the FKBP-L (poly)peptides of the invention also include chemically modified peptides or isomers and racemic forms.

Embodiments of the present invention comprise an isolated FKBP-L polypeptide or a biologically active fragment of a FKBP-L polypeptide, or a biologically active derivative of such a FKBP-L polypeptide or fragment for use as a medicament for treatment of the ocular diseases described herein.

Preferred, but non-limiting, embodiments of the present invention comprise use of a FKBP-L peptide or nucleotide that encodes a FKBP-L peptide as described herein, wherein the FKBP-L polypeptide comprises the amino acid sequence shown in SEQ ID NO:3 (IRQQPRDPPTETLELEVSPDPAS), or an amino acid sequence at least 90% identical to the amino acid sequence shown in SEQ ID NO:3.

As described herein, the methods and pharmaceutical compositions for use according to the present invention may utilize a full-length FKBP-L polypeptide, or biologically active fragments of the polypeptide. Thus, certain embodiments of the present invention comprise a FKBP-L derivative which comprises or consists of a biologically active portion of the N-terminal amino acid sequence of naturally occurring FKBP-L. This sequence may comprise, consist essentially of, or consist of an active N-terminal portion of the FKBP-L polypeptide. In alternate embodiments, the polypeptide may comprise, consist essentially of, or consist of amino acids 1 to 57 of SEQ ID NO: 2 (i.e., SEQ ID NO: 8), or amino acids 34-57 of SEQ ID NO:2 (i.e., SEQ ID NO: 4), or amino acids 35-57 of SEQ ID NO:2 (i.e. SEQ ID NO:3). Or, the peptide may comprise, consist essentially of, or consist of a sequence that comprises at least 18 contiguous amino acids of SEQ ID NO: 4 (e.g., SEQ ID NOs: 10, 12, or 19). In alternate embodiment, the polypeptide used in the methods and compositions of the present invention may comprise, consist essential of, or consist of one of the amino acid sequences shown in any one of SEQ ID NOs: 1-23. In certain embodiments, the present invention comprises a biologically active fragment of FKBP-L, wherein said polypeptide includes no more than 200 consecutive amino acids of the amino acid sequence shown in SEQ ID NO:1, or SEQ ID NO:2, with the proviso that said polypeptide includes the amino acid sequence shown as SEQ ID NO:3.

As described herein, the peptides may be modified (e.g., to contain PEG and/or His tags or other modifications). Or, the present invention may comprise isolated polypeptides having a sequence at least 70%, or 75%, or 80%, or 85%, or 90%, or 95%, or 96%, or 97%, or 98%, or 99% identical to the amino acid sequences as set forth in any one of SEQ ID NOS: 1-23, including in particular sequences at least 70%, or 75%, or 80%, or 85%, or 90%, or 95%, or 96%, or 97%, or 98%, or 99% identical to the amino acid sequence shown as SEQ ID NO:3. In this regard, deliberate amino acid substitutions may be made in the peptide on the basis of similarity in polarity, charge, solubility, hydrophobicity, or hydrophilicity of the residues, as long as the specific biological activity (i.e. function) of the peptide is retained.

The FKBP-L peptide may be of variable length as long as it retains its biological activity and can be used according to the various aspects of the invention described above.

### Fragments of FKBP-L

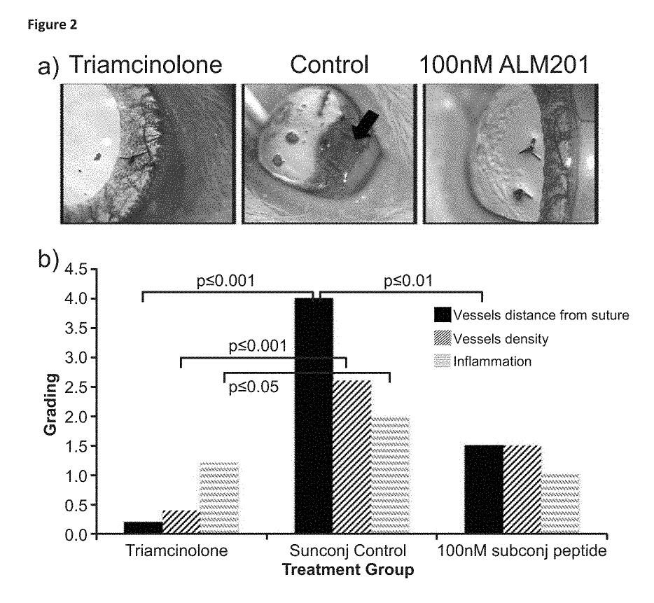

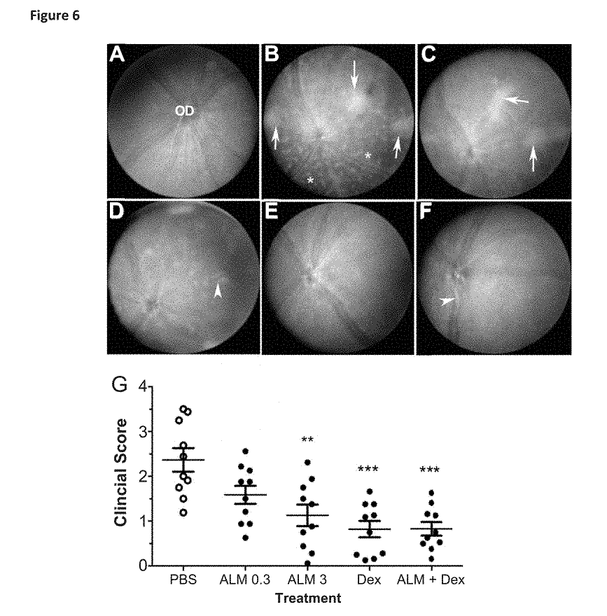

Embodiments of the present invention recognize that certain regions of the N-terminus of the FKBP-L protein may display biological activity, therefore the invention encompasses use of biologically active fragments of FKBP-L, in particular any fragment which exhibits biological activity substantially equivalent to that of the 23-mer peptide (SEQ ID NO:3). In certain embodiments, the biological activity of the FKBP-L 23mer peptide (SEQ ID NO:3; referred to herein also as ALM201) is exhibited as a reduction in blood vessel formation in a rat corneal suture model (FIGS. 2-4). In further embodiments, the biological activity of the FKBP-L 23mer peptide (SEQ ID NO:3; referred to herein also as ALM201) is exhibited as a reduction in retinal inflammation in EAU mice (FIG. 6).

A “fragment” of a FKBP-L polypeptide means an isolated peptide comprising a contiguous sequence of at least 6 amino acids, preferably at least 10 amino acids, or at least 15 amino acids, or at least 20 amino acids, or at least 23 amino acids of FKBP-L. The “fragment” preferably contains no more than 50, or no more than 45, or no more than 40, or no more than 35, or no more than 30, or no more than 25, or no more than 23 contiguous amino acids of FKBP-L. Preferred fragments for use according to the invention are those having the amino acid sequences shown in any one of SEQ ID Nos: 4-23, or minor sequence variants thereof (e.g. variants containing one or more conservative amino acid substitutions).

### Derivatives

An FKBP-L derivative for use in the invention includes polypeptides modified by varying the amino acid sequence of FKBP-L, e.g. SEQ ID NO:1, SEQ ID NO: 2, or SEQ ID NO:29, or a fragment thereof, or a polypeptide at least 70%, 75%, 80%, 85%, 90%, 95%, 96%, 97%, 98%, or 99% identical thereto, or such peptides that have be modified by the addition of a functional group (e.g., PEG). Generation of such peptides may be performed by manipulation of the nucleic acid encoding the polypeptide or by altering the protein itself.

FKBP-L derivatives include analogues of the natural FKBP-L amino acid sequence and may involve insertion, addition, deletion and/or substitution of one or more amino acids, while providing a polypeptide capable of effecting similar biological effects. Also included in the FKBP-L derivatives of the present invention are polypeptides derived from SEQ ID Nos: 1-23.

Thus, FKBP-L derivatives used in the methods and compositions of the present invention also include fragments, portions or mutants of the naturally occurring FKBP-L. In certain embodiments, such derivatives involve the insertion, addition, deletion and/or substitution of 5 or fewer amino acids, more preferably of 4 or fewer, even more preferably of 3 or fewer, most preferably of 1 or 2 amino acids only.

FKBP-L derivatives also include multimeric peptides comprising the FKBP-L polypeptides of SEQ ID NOs: 1-23, and prodrugs including such sequences. For example, in certain embodiments FKBP-L or fragments of FKBP-L may form multimers by the formation of disulfide bonds between monomers.

Derivatives of the FKBP-L polypeptides may include the polypeptide linked to a coupling partner, e.g., an effector molecule, a label, a drug, a toxin and/or a carrier or transport molecule. Techniques for coupling the polypeptides of the invention to both peptidyl and non-peptidyl coupling partners are well known in the art.

FKBP-L derivatives also include fusion peptides. For example, derivatives may comprise polypeptide peptides of the invention linked, for example, to antibodies that target the peptides to diseased tissue, for example, the cornea or retina.

The FKBP-L polypeptide or their analogues may be fused with the constant domain of immunoglobulins (IgA, IgE, IgG, IgM), or portions thereof (CH1, CH2, CH3, or any combination thereof), resulting in chimeric polypeptides. These fusion polypeptides or proteins can facilitate purification and may show an increased half-life in vivo. Such fusion proteins may be more efficient in binding and neutralizing other molecules than monomeric polypeptides or fragments thereof alone. See, e.g., Fountoulakis et al., J. Biochem., 270:3958-3964 (1995).

Fusion proteins of the invention also include FKBP-L polypeptides fused with albumin, for example recombinant human serum albumin or fragments or variants thereof (see, e.g., U.S. Pat. No. 5,876,969, EP Patent 0413622 and U.S. Pat. No. 5,766,883).

The use of polynucleotides encoding such fusion proteins described herein is also encompassed by the invention. The use of a polynucleotide fused to a cytotoxic agent is also encompassed by the invention. In this instance the FKBP-L polypeptide may bind to a receptor and the cytotoxic drug could be internalised.

For example, in alternate embodiments, derivatives may include: site-specific PEGylation (or the like) of peptide to increase half life; or incorporation unnatural amino acids and back bone modifications to stabilize against proteolysis; or cyclic derivatives (to provide proteolytic resistance); or to block the N- and C-termini to prevent or reduce exopeptidase and/or proteinase activity; or to join together multiple copies of peptides either in a contiguous chain via linkers chain or in a dendrimer type of approach to increase ‘avidity’ with cell surface CD44. For example, trimeric covalently linked derivatives of 24mer may be used as derivatives of FKBP-L. Or, the FKBP-L 24mer may be attached to a domain which homotrimerises to form non-covalent trimers. Or, biotin derivatives of peptides which will form tetrameric complexes with streptavidin may be used as derivatives of FKBP-L. Or, FKBP-L or fragments of FKBP-L may form multimers by the formation of disulphide bonds between monomers. In addition, FKBP-L may form oligomers through non-covalent associations, possibly through the predicted tetratricopeptide repeat domains within the protein sequence.

### Reverse Peptide Analogues

Analogues for use in the present invention further include reverse- or retro-analogues of natural FKBP-L proteins, portion thereof or their synthetic derivatives. See, for example, EP 0497 366, U.S. Pat. No. 5,519,115, and Merrifield et al., 1995, PNAS, 92:3449-53, the disclosures of which are herein incorporated by reference. As described in EP 0497 366, reverse peptides are produced by reversing the amino acid sequence of a naturally occurring or synthetic peptide. Such reverse-peptides may retain the same general three-dimensional structure (e. g., alpha-helix) as the parent peptide except for the conformation around internal protease-sensitive sites and the characteristics of the N- and C-termini. Reverse peptides are purported not only to retain the biological activity of the non-reversed “normal” peptide but may possess enhanced properties, including increased biological activity. (See Iwahori et al., 1997, Biol. Pharm. Bull. 20: 267-70). Derivatives for use in the present invention may therefore comprise reverse peptides of natural and synthetic FKBP-L proteins.

Peptides (including reverse peptides and fragments of either) for use in the invention may be generated wholly or partly by chemical synthesis or by expression from nucleic acid. The peptides for use in the present invention can be readily prepared according to well-established, standard liquid or, preferably, solid-phase peptide synthesis methods known in the art (see, for example, J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984).

### Multimeric Peptides

As described above, the peptides may be in the form of multimers. Thus multimers of 2, 3 or more individual FKBP-L polypeptide monomeric units, or two or more fragments of FKBP-L, are within the scope of the invention.

In one embodiment, such multimers may be used to prepare a monomeric peptide by preparing a multimeric peptide that includes the monomeric unit, and a cleavable site (i.e., an enzymatically cleavable site), and then cleaving the multimer to yield a desired monomer.

In one embodiment, the use of multimers can increase the binding affinity for a receptor.

The multimers can be homomers or heteromers. As used herein, the term homomer, refers to a multimer containing only polypeptides corresponding to a specific amino acid sequence (e.g., SEQ ID NO: 1, SEQ ID NO: 2, or SEQ ID NO: 3), or variants, splice variants, fusion proteins, or other FKBP-L analogues or derivatives described herein. These homomers may contain FKBP-L peptides having identical or different amino acid sequences. For example, the multimers can include only FKBP-L peptides having an identical amino acid sequence, or can include different amino acid sequences. The multimer can be a homodimer (e.g., containing only FKBP-L peptides, these in turn may have identical or different amino acid sequences), homotrimer or homotetramer.

As used herein, the term heteromer refers to a multimer containing one or more heterologous polypeptides (i.e., non-FKBP-L peptide or polypeptides) in addition to the FKBP-L (poly)peptides described herein.

The multimers may be the result of hydrophobic, hydrophilic, ionic and/or covalent associations and/or may be indirectly linked, by for example, liposome formation. Thus, in one embodiment, multimers are formed when the FKBP-L peptides described herein contact one another in solution. In another embodiment, heteromultimers are formed when FKBP-L and non-FKBP-L (poly)peptides contact antibodies to the (poly)peptides described herein (including antibodies to the heterologous (poly)peptide sequence in a fusion protein described herein) in solution. In other embodiments, multimers described herein may be formed by covalent associations with and/or between the FKBP-L peptides (and optionally non-FKBP-L peptides) described herein.

Such covalent associations can involve one or more amino acid residues contained in the FKBP-L sequence (e.g., that recited in SEQ ID NOs: 1-23). In one embodiment, the covalent associations are the consequence of chemical or recombinant manipulation. Alternatively, such covalent associations can involve one or more amino acid residues contained in the heterologous polypeptide sequence in a FKBP-L fusion protein. In one example, covalent associations are between the heterologous sequence contained in a fusion protein described herein (see, e.g., U.S. Pat. No. 5,478,925). In another specific example, covalent associations of fusion proteins described herein are using heterologous polypeptides sequence from another protein that is capable of forming covalently associated multimers, for example, oesteoprotegerin (see, e.g., International Publication NO: WO 98/49305). In another embodiment, two or more polypeptides described herein are joined through peptide linkers. Examples include those peptide linkers described in U.S. Pat. No. 5,073,627. Proteins comprising multiple FKBP-L peptides separated by peptide linkers can be produced using conventional recombinant DNA technology.

Multimers may also be prepared by fusing the FKBP-L (poly)peptides to a leucine zipper or isoleucine zipper polypeptide sequence. Among the known leucine zippers are naturally occurring peptides and derivatives thereof that dimerize or trimerize. Examples of leucine zipper domains suitable for producing soluble multimeric proteins described herein are those described in PCT application WO 94/10308. Recombinant fusion proteins comprising a polypeptide described herein fused to a polypeptide sequence that dimerizes or trimerizes in solution can be expressed in suitable host cells, and the resulting soluble multimeric fusion protein can be recovered from the culture supernatant using techniques known in the art.

The multimers may also be generated using chemical techniques known in the art. For example, polypeptides to be contained in the multimers described herein may be chemically cross-linked using linker molecules and linker molecule length optimisation techniques known in the art (see, e.g., U.S. Pat. No. 5,478,925). Additionally, the multimers can be generated using techniques known in the art to form one or more inter-molecule cross-links between the cysteine residues located within the sequence of the polypeptides desired to be contained in the multimer (see, e.g., U.S. Pat. No. 5,478,925). Further, polypeptides described herein may be routinely modified by the addition of cysteine or biotin to the C-terminus or N-terminus of the polypeptide and techniques known in the art may be applied to generate multimers containing one or more of these modified polypeptides (see, e.g., U.S. Pat. No. 5,478,925). Additionally, techniques known in the art can be used to prepare liposomes containing two or more C-12-C peptides desired to be contained in the multimer (see, e.g., U.S. Pat. No. 5,478,925).

Alternatively, those multimers including only naturally-occurring amino acids can be formed using genetic engineering techniques known in the art. Alternatively, those that include post-translational or other modifications can be prepared by a combination of recombinant techniques and chemical modifications. In one embodiment, the FKBP-L peptides are produced recombinantly using fusion protein technology described herein or otherwise known in the art (see, e.g., U.S. Pat. No. 5,478,925, which is herein incorporated by reference in its entirety). For example, polynucleotides coding for a homodimer described herein can be generated by ligating a polynucleotide sequence encoding a FKBP-L peptide described herein to sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925). The recombinant techniques described herein or otherwise known in the art can be applied to generate recombinant FKBP-L (poly)peptides that contain a transmembrane domain (or hydrophobic or signal peptide) and that can be incorporated by membrane reconstitution techniques into liposomes (see, e.g., U.S. Pat. No. 5,478,925).

### Pro-Drugs

The polypeptides described herein are intended, at least in some embodiments, to be administered to a human or other mammal to treat or prevent an ocular disorder, e.g. corneal neovascularisation, or an inflammatory disease of the eye. As discussed below, peptides are typically administered to the eye topically, via subconjunctival injection, intrastromal injection or any other route of intraocular injection, or even via systemic administration, e.g. oral administration or a parenteral route, such as intraperitoneal administration.

Peptides or polypeptides can be conjugated to various moieties, such as polymeric moieties, to modify the physiochemical properties of the peptide drugs, for example, to increase resistance to acidic and enzymatic degradation and to enhance penetration of such drugs across mucosal membranes. For example, Abuchowski and Davis have described various methods for derivatizating enzymes to provide water-soluble, non-immunogenic, in vivo stabilized products (“Soluble polymers-Enzyme adducts,” Enzymes as Drugs, Eds. Holcenberg and Roberts, J. Wiley and Sons, New York, N.Y. (1981)).

Thus, in certain embodiments, the FKBP-L peptides may be conjugated to polymers, such as dextrans, polyvinyl pyrrolidones, glycopeptides, polyethylene glycol and polyamino acids. The resulting conjugated polypeptides retain their biological activities and solubility in water for parenteral applications. In an embodiment, the FKBP-L peptides may be coupled to polyethylene glycol or polypropropylene glycol having a molecular weight of 500 to 20,000 Daltons to provide a physiologically active non-immunogenic water soluble polypeptide composition (see e.g., U.S. Pat. No. 4,179,337 and Garman, A. J., and Kalindjian, S. B., FEBS Lett., 1987, 223, 361-365). The polyethylene glycol or polypropylene glycol may protect the polypeptide from loss of activity and the composition can be injected into the mammalian circulatory system with substantially no immunogenic response. In other embodiments, the FKBP-L is coupled to an oligomer that includes lipophilic and hydrophilic moieties (see e.g., U.S. Pat. Nos. 5,681,811, 5,438,040 and 5,359,030).

Prodrugs can be prepared for example, by first preparing a maleic anhydride reagent from polydispersed MPEG5000 and then conjugating this reagent to the polypeptides disclosed herein. The reaction of amino acids with maleic anhydrides is well known. The hydrolysis of the maleyl-amide bond to reform the amine-containing drug is aided by the presence of the neighbouring free carboxyl group and the geometry of attack set up by the double bond. The peptides can be released (by hydrolysis of the prodrugs) under physiological conditions.

The polypeptides can also be coupled to polymers, such as polydispersed PEG, via a degradable linkage, for example, the degradable linkage shown (with respect to pegylated interferon) in Roberts, M. J., et al., Adv. Drug Delivery Rev., 2002, 54, 459-476.

The polypeptides can also be linked to polymers such as PEG using 1,6 or 1,4 benzyl elimination (BE) strategies (see, for example, Lee, S., et al., Bioconjugate Chem., (2001), 12, 163-169; Greenwald, R. B., et al., U.S. Pat. No. 6,180,095, 2001; Greenwald, R. B., et al., J. Med. Chem., 1999, 42, 3657-3667.); the use of trimethyl lock lactonization (TML) (Greenwald, R. B., et al., J. Med. Chem., 2000, 43, 475-487); the coupling of PEG carboxylic acid to a hydroxy-terminated carboxylic acid linker (Roberts, M. J., J. Pharm. Sci., 1998, 87(11), 1440-1445), and PEG prodrugs involving families of MPEG phenyl ethers and MPEG benzamides linked to an amine-containing drug via an aryl carbamate (Roberts, M. J., et al., Adv. Drug Delivery Rev., 2002, 54, 459-476), including a prodrug structure involving a meta relationship between the carbamate and the PEG amide or ether (U.S. Pat. No. 6,413,507 to Bently, et al.); and prodrugs involving a reduction mechanism as opposed to a hydrolysis mechanism (Zalipsky, S., et al., Bioconjugate Chem., 1999, 10(5), 703-707).

The FKBP-L polypeptides of the present invention have free amino, amido, hydroxy and/or carboxylic groups, and these functional groups can be used to convert the peptides into prodrugs. Prodrugs include compounds wherein an amino acid residue, or a polypeptide chain of two or more (e.g., two, three or four) amino acid residues which are covalently joined through peptide bonds to free amino, hydroxy or carboxylic acid groups of various polymers, for example, polyalkylene glycols such as polyethylene glycol. Prodrugs comprising the polypeptides of the invention or pro-drugs from which peptides of the invention (including analogues and fragments) are released or are releaseable are considered to be analogues of the invention.

Prodrugs also include compounds wherein PEG, carbonates, carbamates, amides and alkyl esters which are covalently bonded to the above peptides through the C-terminal carboxylic acids. Thus, embodiments of the present invention comprise site-specific PEG addition.

### Treatment/Prevention of Corneal Neovascularisation

The examples of the present application conclusively demonstrate that topical administration of FKBP-L peptide (specifically the peptide of SEQ ID NO:3) to the surface of the cornea can dramatically reduce blood vessel formation. In addition, FKBP-L peptide (specifically the peptide of SEQ ID NO:3) also act as an anti-inflammatory agent, with an efficacy superior to existing drug treatments in the rat corneal suture model. This result clearly supports the utility of FKBP-L polypeptide and peptide fragments thereof in the treatment/prevention of corneal neovascularisation.

Various ocular disorders are associated with and/or mediated by corneal neovascularisation, and may be treated using the FKBP-L peptide compounds, compositions and methods described herein. Corneal neovascular disease is characterized by invasion of new blood vessels into the corneal tissue and is a common cause of blindness. Other diseases associated with corneal neovascularisation include, but are not limited to, epidemic keratoconjunctivitis, Vitamin A deficiency, atopic keratitis, superior limbic keratitis, pterygium keratitis sicca, periphigoid radial keratotomy, and corneal graft rejection.

The current treatment for neovascularization-induced corneal opacity damage is corneal graft; however, the presence of neovascularization itself contributes to a worse prognosis following grafting in this high-risk group. When corneal grafts are placed into an avascular recipient bed (low-risk keratoplasty), 2-year graft survival rates approach 90% under cover of topical steroids (Küchle et al., 2002). In vascularized, recipient beds (high-risk keratoplasty) the graft survival for 2 years decreases significantly below 50% (Cursiefen et al., 2002). Sutures invariably induce a significant neovascular response, which is also associated with an increased risk of corneal rejection; therefore even the means by which a corneal graft is fastened in place by itself increases the risk of graft rejection. New therapeutic options, such as those presented in this application, are desperately needed, as currently available treatments for this type of eye disease, such as corticosteroids, have limited efficacy and a risk of side effects. Therefore, a particularly important aspect of the invention relates to prevention and/or treatment of corneal neovascularisation in patients who have undergone corneal graft surgery. It is contemplated that treatment with FKBP-L peptide may be administered prior to corneal graft surgery and/or post-surgery in order to reduce the risk of graft rejection.

Corneal vascularisation also often occurs after corneal stromal infection from the herpes virus, usually secondary to reactivation of the virus (after an initial primary infection) from its latent position within the trigeminal ganglion (Liu et al., 2006). Many factors have been implicated in the induction of the neovascularisation process, which can also change during the evolving course of the disease pathogenesis (Suryawanshi et al., 2011), but most recent work has focused upon VEGF-A. This growth factor is produced both by directly infected cells and also by bystander cells and infiltrating inflammatory cells induced to produce it via a variety of paracrine factors including 11-6 (Kanangat et al., 1996). During HSV-1 infection, inflammation and angiogenesis trigger each other, with the newly formed leaky blood vessels which lack pericytes and separated endothelial cells releasing inflammatory cells and cytokines into the corneal stroma, this has the effect of inducing the ingrowth of more blood vessels to compound the problem (Azar, 2006).

Most treatment strategies to induce regression of blood vessels within the cornea have utilized anti VEGF treatments. This type of treatment is often quite successful if it occurs early in the condition, however once blood vessels are well established they become unresponsive to anti VEGF treatments. The difficulties with any of the current anti VEGF approaches are that they often require multiple injections over a long period, particularly as the condition often waxes and wanes producing more inflammation and promoting further vascularisation. Currently no medical treatment is available to remove blood vessels, which are well established within the cornea. The only treatments in these circumstances are through the use of either laser treatments or cautery, both of which are far from satisfactory often requiring multiple surgical interventions (Gupta & Illingworth, 2011).

A particular (surprising) benefit of the use of FKBP-L peptide (e.g. the peptide of SEQ ID NO:3) in the treatment/prevention of corneal neovascularisation is that reduction in blood vessel formation is also combined with an anti-inflammatory effect. It is also highly beneficial that such effects are achieved following topical administration of the peptide, although it is also envisaged that the peptide may be administered via other routes, such as subconjunctival injection, or other modes of intraocular injection.

As well as treating/preventing corneal neovascularisation, topical administration of FKBP-L peptide (e.g. the peptide of SEQ ID NO:3) may be effective at treating/preventing indications associated with neovascularisation elsewhere in the eye, due to the penetration of the peptide to all compartments of the eye. Diseases associated with ocular neovascularisation that may be treated/prevented by administration (e.g. topical administration) of FKBP-L peptide (e.g. the peptide of SEQ ID NO:3) include, for example, retinopathy of prematurity (ROP), diabetic retinopathy, neovascular age-related macular degeneration, sickle cell retinopathy, and/or retinal vein occlusion.

### Treatment/Prevention of Inflammation of the Eye

The examples of the present application also conclusively demonstrate that administration of FKBP-L peptide (specifically the peptide of SEQ ID NO:3) suppressed retinal inflammation in a mouse model of experimental autoimmune uveitis (EAU) in a dose-dependent manner. In addition, as reported above, topical administration of FKBP-L peptide also produces an anti-inflammatory effect in a rat corneal suture model. For example, topical administration of FKBP-L peptide (SEQ ID NO: 3) reduces both total and inflammatory cell infiltrates (measured using, for example, CD68+ or CD44+ cells) in such a model. Together, these results clearly support the utility of FKBP-L polypeptide and peptide fragments thereof in the treatment/prevention of inflammatory diseases of the eye, particularly diseases associated with/mediated by inflammation of the retina and/or inflammation of the cornea. Furthermore, topical administration of FKBP-L peptide (SEQ ID NO: 3) results in the penetration of the peptide to all layers of the eye. FKBPL peptide thus has the potential to treat inflammatory diseases of the eye associated with/mediated by inflammation of other elements of the eye, for example inflammation of the choroid, sclera and/or ocular muscle.

Examples of inflammatory eye diseases which may be treated/prevented by administration of FKBP-L polypeptides and peptide fragments thereof, in particular the peptide of SEQ ID NO:3, include (but are not limited to) uveitis, dry eye syndrome, blepharokeratoconjunctivitis and various forms of keratitis.

### Formulations/Routes of Administration

As used herein, “treatment” or “therapy” includes any regime that can benefit a human or non-human animal. The treatment may be in respect of an existing condition or may be prophylactic (preventative treatment). Treatment may include curative, alleviation or prophylactic effects.

Corneal neovascularisation can be reduced by administering an effective amount of a FKBP-L polypeptide, or peptide fragment thereof, to a patient in need of such treatment.

The dose of the FKBP-L polypeptide administered may vary depending upon the precise nature of the disorder being treated. In alternate embodiments, a dosage to be achieved in vivo would be equivalent to an in vitro level of greater than 10⁻¹² M, or 10⁻¹¹ M, or 10⁻¹⁰ M, or 10⁻⁹ M, or 10⁻⁸ M, or 10⁻⁷ M, or 10⁻⁶ M, or 10⁻⁵ M. Thus, a dosage to be achieved in vivo may be equivalent to an in vitro level of 10⁻¹² M to 10⁻⁵ M, or 10⁻¹¹ M to 10⁻⁶ M, or 10⁻¹⁰ M to 10⁻⁷ M, or 10⁻⁹ M to 10⁻⁷ M or ranges therein. In alternate embodiments, the dosage used may be equivalent to an in vitro level of about 1-10000 ng/ml, or about 10-5000 ng/ml, or about 100-1000 ng/ml. Or, in certain embodiments, the dosage may comprise from about 0.00001 to 500 mg/kg/day, or from about 0.0001 to 300 mg/kg/day, or from about 0.003 to 100 mg/kg/day, or from about 0.03 to 30 mg/kg/day, or from about 0.1 mg/kg/day to 10 mg/kg/day, or from about 0.3 mg/kg/day to 3 mg/kg/day. In further alternate embodiments, the human in vivo dosage used may be equivalent to a rat in vivo dosage of from 10⁻⁹ M to 10⁻⁸ M. In further alternate embodiments, the human in vivo dosage used may be from 10⁻¹⁰ to 10⁻⁹ or from 10⁻⁸ to 10⁻⁶ M. In certain embodiments the human in vivo dosage may be from 10⁻⁹M to 10⁻⁸ M.

The route of administration may also vary depending upon the precise nature of the disorder being treated. Suitable routes of administration may include, but are not limited to, topical application to the eye, e.g. topical administration to the surface of the cornea or to other external surfaces of the eye, intraocular injection, subconjunctival injection, intravitreal injection, intraperitoneal administration, oral administration.

For administration to a human subject, the FKBP-L polypeptide or peptide fragment thereof may be formulated into a pharmaceutical composition/dosage form suitable for administration via the chosen delivery route.

It is envisaged that the FKBP-L polypeptide or peptide fragment thereof may be used as a stand-alone treatment, or as a component of a combination treatment.

An embodiment of the invention relates to a combination treatment comprising the combination of FKBP-L polypeptide or peptide fragment thereof, and in particular the peptide of SEQ ID NO:3, with dexamethasone (or any derivative or analogue thereof), for the treatment of inflammatory diseases of the eye (e.g. uveitis, dry eye syndrome or blepharokeratoconjunctivitis).

### EXAMPLES

### Example 1—Effect of ALM201 on Neovascularisation on the Ocular Surface

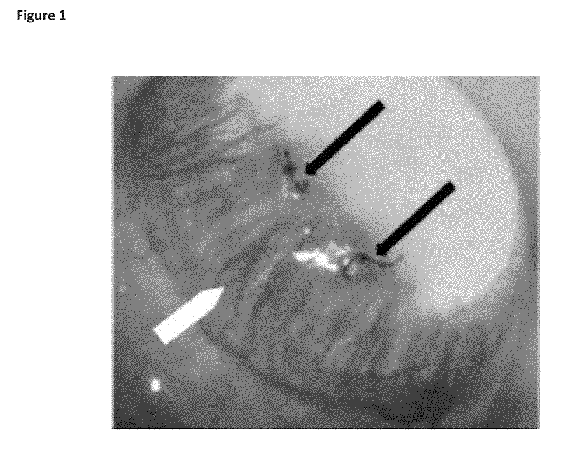

To assess the action of ALM201 (SEQ ID NO:3) on the ocular surface, the ability to halt vessel growth was measured. Vessel growth was stimulated through a well-accepted model (Shi et al., 2011) whereby a suture was placed in the central cornea of rat eyes (FIG. 1). We have shown the rat eye is similar anatomically to the human eye (Moore J E, McMullen C B, Mahon G and Adamis A P. The corneal epithelial stem cell. DNA Cell Biol. 2002; 21(5-6):443-51; incorporated herein by reference).

### Experimental Groups

The experimental groups were:
Subconjunctival injection of peptide (5 rats), control group (5 rats) and positive control group (5 rats)
Intrastromal injection of peptide (5 rats), control group (5 rats) and positive control group (5 rats)
Topical application of peptide (5 rats), control group (5 rats) and positive control group (5 rats)

Methods

A suture was placed in the central cornea of one eye of each animal while the control eye remained untouched. For the injection groups, peptide was added to the rat's eyes at day 0, day 3, and day 6. For the topical application group, peptide and the positive control was added daily. Each day after suture, rats were monitored for vessel growth and photographed using a Hawk Eye Portable Digital Slit Lamp (Dioptrix, France) and inverted microscope (Nikon's E600FN; Surrey, UK). At a time point when a difference is noted (e.g. day 6) between untreated and peptide treated animals, they received 8 μg/g tail vein injections of an endothelial-specific fluorescein-conjugated lectin (Lycopersicon esculentum; Vector Laboratories, Burlingame, Calif.). Thirty minutes later, the eyes were harvested and fixed with 10% neutral buffered formalin for 24 hours. The corneas were isolated and flat-mounted on glass slides. The fluorescence in the perfused vessels was captured using a Leica SP5 multiphoton microscope (Milton Keynes, UK). Vessel formation within tagged information file format (.tiff) files was measured by visualization with Image J (Rasband, 1997-2014).

At day 6 all rats were graded by an opthalmologist blinded to the treatment. Grading scales were as follows:

Vessel distance from suture: 0=no reach; 1=small distance; 2=moderate distance; 3=¾ of the way; 4=reached sutures;

Vessel density grading: 0=no density; 1=mild; 2=moderate; 3=high Inflammation grading: 0=none; 1=minimal; 2=moderate; 3=severe;

Results

Subconjunctival Injection

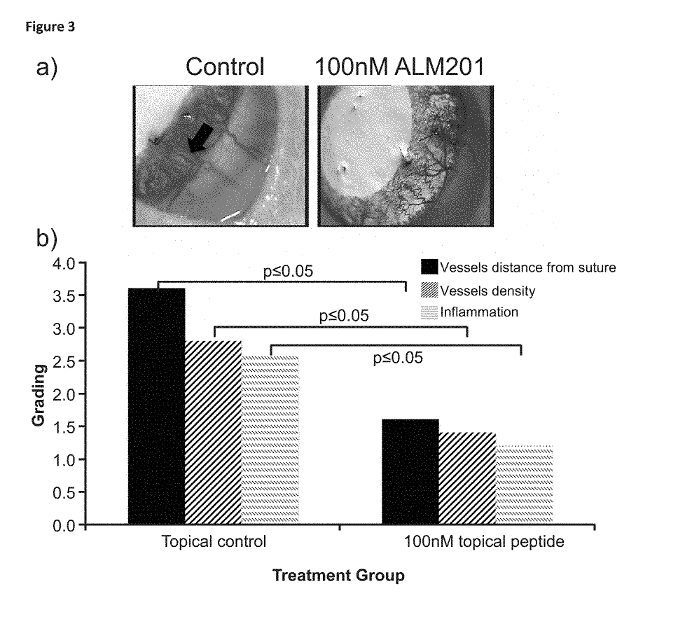

For statistical analysis, both Triamcinolone and 100 nM ALM201 treatments were compared to the PBS control treatment. The neovascularisation induced by the sutures is shown in the control image in FIG. 3 by a black arrow; these vessels grow in a straight line towards the sutures and differ from the normal vasculature seen in the Triamcinolone and ALM201 treatments (FIG. 2(a)). A significant difference of p<0.01 was shown for 100 nM ALM201 vessel distance from suture when compared to the PBS control (FIG. 2(b)).

Topical Treatment

The black arrow in the control image demonstrates neovasculature while the ALM201 image demonstrates normal vasculature within the rat eye (FIG. 3(a)). Vessel distance from suture, vessel density and inflammation were all significantly different in the ALM201 treatment compared to the PBS control treatment (FIG. 3(b)).

Subconjunctival Injection Using Hooded Lister Rats

Images of vasculature in animals with non-pigmented eyes can be difficult to see. The experiment with topical ALM201 was repeated in Hooded Lister Rats to provide clearer pictures of the anti-vascular action.

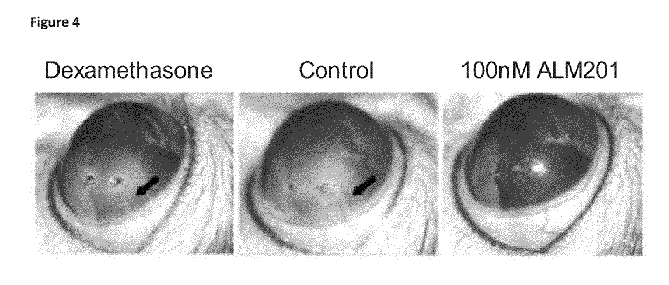

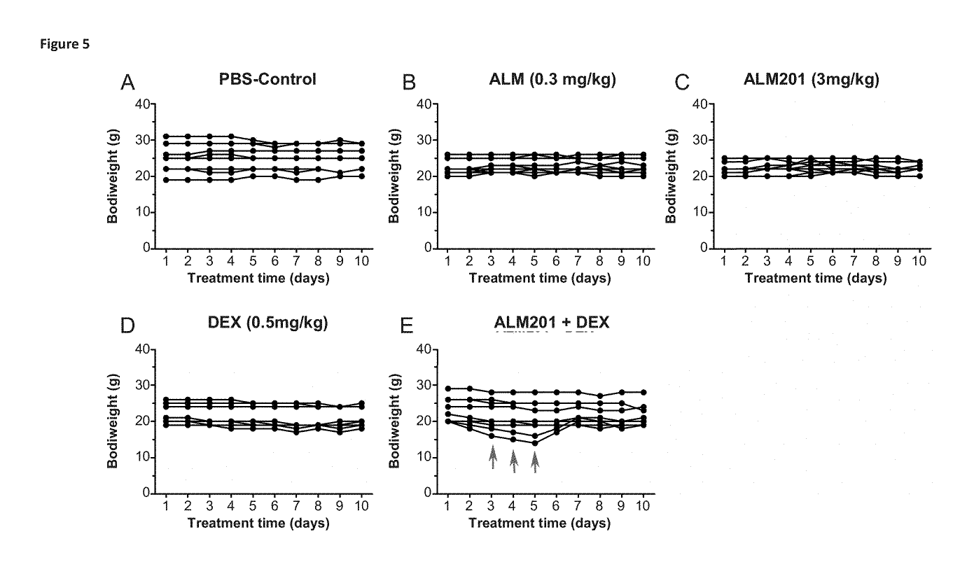

In FIG. 4 neovascularisation induced by the sutures is shown by a black arrow in the control image (centre panel) and dexamethasone treated rats (left panel). These vessels grow in a straight line towards the sutures. In the dexamethasone treated rats, the neovascularisation is not as marked as that viewed in the control image. There is an absence of neovascularisation within the 100 nM ALM201 treatment image (right panel).

Conclusions

ALM201 (SEQ ID NO:3) prevented growth of new blood vessels after corneal damage in the suture model after subconjunctival injection or topical application. The peptide also had anti-inflammatory activity when injected. Inflammation was not assessed after topical application. These initial experiments show clear positive effects of ALM201 in reducing neovascularisation after corneal damage indicating ALM201 could be effective in treating a number of eye conditions.

### Example 2—Effect of ALM201 on Autoimmune Uveitis

The effect of ALM201 on autoimmune uveitis was tested in a mouse model of experimental autoimmune uveitis (EAU).

Methods

Animals

Sixty C57BL/6J mice (38 female, 22 male, 9-12 weeks old) were purchased from the Biological Resource Unit (BRU) at Queen's University Belfast. All mice were maintained in a normal experimental room and exposed to a 12-hour-dark-12-hour light cycle. All procedures concerning the use of animals in this study were performed according to the Association for Research in Vision and Ophthalmology (ARVO) Statement for the Use of Animals in Ophthalmic and Vision Research, and under the regulations of the United Kingdom Animal (Scientific Procedure) 1986.

Induction of EAU

Mice were immunised with human Interphotoreceptor Retinoid Binding Protein (IRBP) peptide 1-20 (GPTHLFQPSLVLDMAKVLLD, GL Biochem, Shanghai, China) using a protocol described previously (Chen M., Copland D. A., Zhao J., Liu J., Forrester J. V., Dick A. D., Xu H., 2012. Persistent inflammation subverts thrombospondin-1-induced regulation of retinal angiogenesis and is driven by CCR2 ligation. Am. J. Pathol. 180, 235-245; Xu H., Manivannan A., Crane I., Dawson R., Liversidge J., 2008. Critical but divergent roles for CD62L and CD44 in directing blood monocyte trafficking in vivo during inflammation. Blood 112, 1166-1174., each incorporated herein by reference). Briefly, 500 mg (100 μl) of IRBP peptide 1-20 emulsified 1:1 in complete Freund's adjuvant (DIFCO Laboratories, Detroit, USA) with an additional 2.5 mg/ml Mycobacterium tuberculosis H37Ra (DIFCO Laboratories) were injected subcutaneously into each mouse. An additional 1 mg Bordetella pertussis toxin (Tocris Bioscience, Bristol, UK) was administered intraperitoneally immediately after peptide injection.

Topic Endoscopic Fundus Imaging (TEFI)

Mouse pupils were dilated with 1% atropine sulphate and 2.5% phenylephrine hydrochloride (Chauvin, Essex, UK). The animals were anaesthetised by isofluorane. A TEFI system described previously (Paques M., Guyomard J. L., Simonutti M., Roux M. J., Picaud S., Legargasson J. F., Sahel J. A., 2007. Panretinal, high-resolution color photography of the mouse fundus. Invest. Ophthalmol. Vis. Sci. 48, 2769-2774., Xu H., Koch P., Chen M., Lau A., Reid D. M., Forrester J. V., 2008. A clinical grading system for retinal inflammation in the chronic model of experimental autoimmune uveoretinitis using digital fundus images. Exp. Eye Res. 87, 319-326., each incorporated herein by reference) was used to obtain fundus images at days 12, 14, and 24 post-immunisation (p.i.). Images were captured using a Nikon D90 camera and saved in TFPI format. Clinical score of retinal inflammation was accessed using the criteria described previously by us (Xu et al. 2008a).

Group Allocation

Based on clinical score of retinal inflammation, mice were designated into five groups to ensure that the average clinical score was comparable between different groups. Ten mice were assigned into each group. Table 1 shows the average clinical EAU score at day 14 p.i. (the day that the treatment started) in different groups.

TABLE 1

| | | Clinical Score | | |
|---|---|---|---|---|
| | | pre-treatment | | |
| Group | N | (Mean ± SD) | P value * | Treatment |
| 1 | 10 | 1.18 ± 0.82 | NS | PBS |
| 2 | 10 | 1.13 ± 0.95 | NS | ALM201 0.3 |
| | | | | mg/kg |
| 3 | 10 | 0.87 ± 0.54 | NS | ALM201 3 |
| | | | | mg/kg |
| 4 | 10 | 0.85 ± 0.75 | NS | Dexamethasone |
| | | | | 0.5 mg/kg |
| 5 | 10 | 1.09 ± 0.71 | NS | ALM201 0.3 |
| | | | | mg/kg + |
| | | | | Dexamethasone |
| | | | | 0.5 mg/kg |

* One-way ANOVA, followed by Tukey's Multiple Comparison Test.
NS, no significant difference between other groups

Treatments

All animals were treated for 10 days from day 14 to 24 p.i. Below are treatment details:

Group 1: 100 ml PBS, i.p. injection, once daily.

Group 2: ALM201 0.3 mg/kg in 100 ml, i.p. injection, once daily.

Group 3: ALM201 3 mg/kg in 100 ml, i.p. injection, once daily.

Group 4: Dexamethasone 0.5 mg/kg in 100 ml, p.o. (gavage), once daily.

Group 5: ALM201 0.3 mg/kg in 100 ml, i.p. injection+Dexamethasone 0.5 mg/kg in 100 ml, p.o. (gavage) once daily.

Sample Collection and Histopathology

On day 24 p.i., fundus images were taken from all experimental mice using the TEFI system. Mice were then sacrificed by CO2 inhalation and eyes were carefully removed. All eyes were fixed in 2.5% (w/v) glutaraldehyde (Agar Scientific Ltd, Cambridge, UK) for 2 days at room temperature. Eyes were embedded in paraffin for standard H&E staining. Histological scores of retinal inflammation were graded using a standard scoring system described previously (Dick A. D., Cheng Y. F., Liversidge J., Forrester J. V., 1994. Immunomodulation of experimental autoimmune uveoretinitis: a model of tolerance induction with retinal antigens. Eye 8 (Pt 1), 52-59, incorporated herein by reference). Retinal sections from at least three different layers from each eye were used for histopathological analysis. The average score from three layers was used as the final pathological score of the eye.

Statistical Analysis

Data (Clinical score and histological score) was expressed as mean±SD. One way ANOVA followed by the Tukey's Multiple Comparison Test was used to detect difference between all treatment groups. In addition, Mann-Whitney U test (two tails) was used to detect the difference between ALM201 or dexamethasone treated group and PBS control group. Paired Student's t test was used to compare clinical score before and after treatment.

Results

Bodyweight

Bodyweight (BW) of each mouse was monitored daily during the course of treatment. The results show that all mice from the PBS control, ALM201 0.3 mg/kg, ALM 3 mg/kg and Dexamethasone 0.5 mg/kg groups had a stable BW during the 10-day treatment period (FIG. 5A-5D). Two mice from the ALM201+Dexamethasone treatment group had reduced BW at days 3, 4, and 5, but regained BW at day 6 and remained stable at the end of the experiment (FIG. 5E).

Effect in EAU

Clinical Inflammation:

In normal non-immunised mice, optic disc (OD) and retinal blood vessels can be clearly visualised in fundus images (FIG. 6A). Severe inflammation including infiltrations around the OD, multiple large infiltrates around blood vessels (arrows in FIG. 6B), multiple small infiltrates (asterisks, FIG. 6B) and infiltration around retinal blood vessels were observed in the majority of EAU mice treated with PBS at day 24 p.i. (FIG. 6B). Retinal inflammation was also observed in mice receiving 0.3 mg/kg ALM201 treatment, although the number of infiltrates was fewer (FIG. 6C) than that in PBS treated EAU mice. Small whitish sheets around blood vessels (vasculitis) was observed in EAU mice receiving 3 mg/kg ALM201 (FIG. 6D), Dexamethasone (0.5 mg/kg) FIG. 6E) and ALM201 0.3 mg/kg+Dexamethasone (0.5 mg/kg) treatment. Large infiltrates around retinal blood vessels were rarely observed in mice from these groups (FIG. 6D-6F). Clinical score analysis showed that ALM201 dose-dependently suppressed inflammation in EAU (FIG. 6G). Dexamethasone (0.5 mg/kg) strongly suppressed retinal inflammation (FIG. 6G). Although there was no statistical significant difference in terms of clinical score between mice treated with 3 mg/kg ALM201 and 0.5 mg/kg Dexamethasone, the later appears to have lower clinical scores. The combination of 0.5 mg/kg Dexamethasone and ALM201 0.3 mg/kg did not further reduce the clinical score compared to Dexamethasone alone (FIG. 6G).

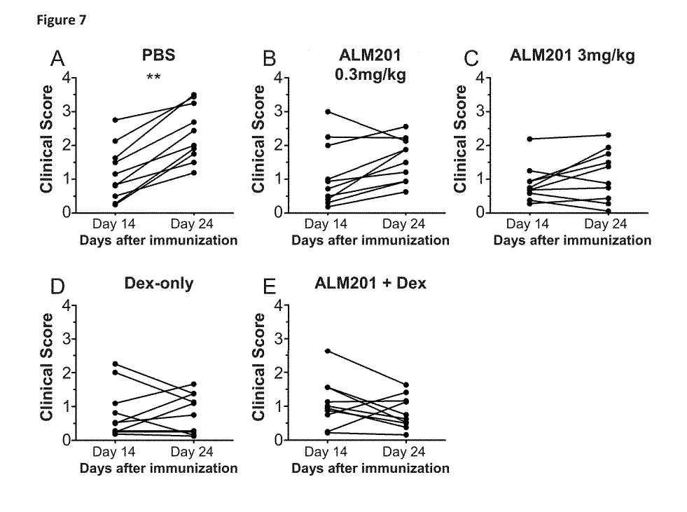

When the clinical scores of the same mouse before (at day 14 p.i.) and after treatment (day 24 p.i.) were compared, all mice in the PBS treated group had increased scores (an average of 1.82 increment in clinical score, FIG. 7A and Table 2), suggesting further development of inflammation from days 14 to 24 p.i.

In mice treated with 0.3 mg/kg ALM201, one mouse had decreased clinical score and one remained unchanged, whereas the other 8 mice experienced slighted increased clinical scores from day 14 to day 24 p.i. (average increment score 0.68. FIG. 7B). However, the overall clinical score did not significant change from day 14 to 24 p.i in this group of mice (FIG. 7B, Table 2). ALM201 3 mg/kg treatment decreased EAU clinical score in 3 mice (average decrement in clinical score: 0.33) and prevented further increment in clinical score in 3 mice (FIG. 7C, table 2), suggesting that this treatment is able to reduce or prevent the development of retinal inflammation in EAU. Four mice had increased clinical score with an average increment of 0.82 point (Table 2, FIG. 7C). Dexamethasone treatment reduced EAU score in 3 mice (average reduction 0.69) and maintained EAU score in 4 mice (FIG. 7D, table 2), and 3 mice had increased EAU score (average increment 0.80) after treatment. The combined therapy of ALM201 and Dexamethasone reduced EAU score in 6 mice (average reduction 0.69) and maintained EAU score in 2 mice (FIG. 7E, table 2). Only two mice experienced progressive retinal inflammation. The results suggest that the combination of Dexamethasone and ALM201 has stronger immune suppressive effect than Dexamethasone or ALM201 alone in this EAU model.

TABLE 2

| | | No. of | | No. of | | | | Difference |
|---|---|---|---|---|---|---|---|---|
| | | mice with | | mice with | | No. of mice | | between |
| | | increased | | decreased | | with no | | day 14 p.i. and |
| Group | Treatment | score (%) | | score (%) | | change (%) | | day 24 p.i. |
| 1 | PBS | 10 | (100%) | 0 | (0%) | 0 | (0%) | Yes (p = 0.01) |
| 2 | ALM201 0.3 mg/kg | 8 | (80%) | 1 | (10%) | 1 | (10%) | No |
| 3 | ALM201 3 mg/kg | 4 | (40%) | 3 | (30%) | 3 | (30%) | No |
| 4 | Dexamethasone | 3 | (30%) | 3 | (30%) | 4 | (40%) | No |
| | 0.5 mg/kg | | | | | | | |
| 5 | ALM201 0.3 mg/kg + | 2 | (20%) | 6 | (60%) | 2 | (20%) | No |
| | Dexamethasone | | | | | | | |
| | 0.5 mg/kg | | | | | | | |

Histopathology:

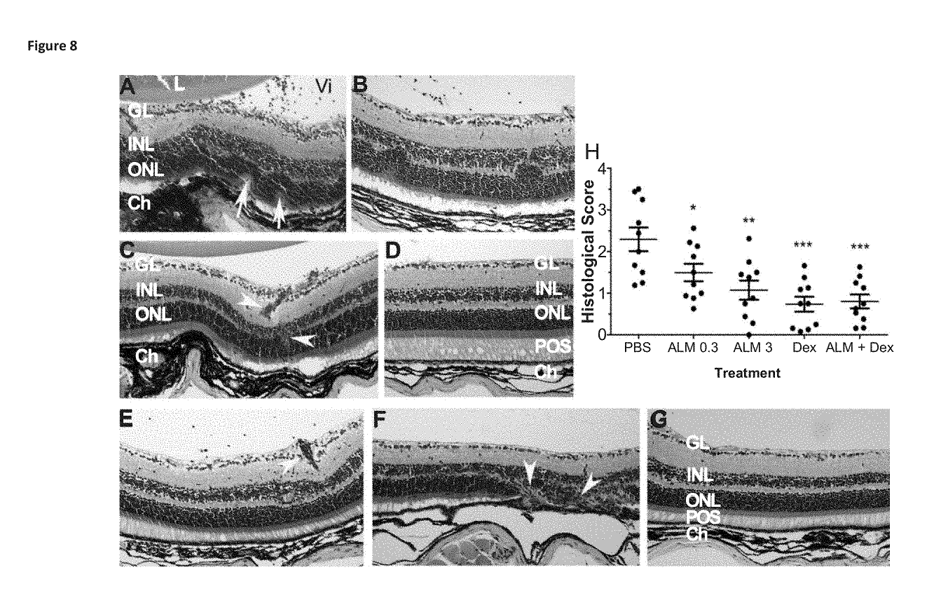

PBS treatment: Marked cell infiltration was observed in the vitreous cavity and the neuroretina of PBS treated control EAU mice (FIG. 8A). Retinal folds were frequently observed (white arrows, FIG. 4A). Photoreceptor outer segments (POS) were absent (FIG. 8A). The normal retinal structure was severely disrupted (FIG. 8A).

ALM201 (0.3 mg/kg) treatment: Fewer infiltrating cells were observed in mice treated with 0.3 mg/kg ALM201 (FIG. 8B). Although granuloma-like lesions (green arrow, FIG. 8B) were occasionally detected, retinal structure was better preserved (FIG. 4B) compared to PBS treated mice (FIG. 8A).

ALM201 (3 mg/kg) treatment: In eyes with severe inflammation prior to treatment, a few infiltrating cells with retinal scars were observed (Arrowheads, FIG. 8C). In eyes with mild inflammation prior to treatment, only few infiltrating cells were observed and retinal structures including POS were largely preserved (FIG. 8D).

Dexamethasone (0.5 mg/kg, FIG. 8E) and Dexamethasone (0.5 mg/kg)+ALM201 0.3 mg/kg (FIG. 8F, 8G) treatment: Similar to ALM201 3 mg/kg treated mice, in eyes with severe inflammation prior to the therapy, scar lesions were observed (arrowheads, FIG. 8E, 8F). A few infiltrating cells were observed in Dexamethasone treated mice (FIG. 8E). Retinal layers including POS were preserved (apart from the areas with scars). In eyes with mild inflammation prior to therapy, infiltrating cells were rarely detected, and no significant structural damages were observed (FIG. 8G). When retinal inflammation was graded using a standard histological scoring system, which takes into account of the number/area of immune cell infiltration and retinal structural damage, ALM201 dose-dependently suppressed retinal inflammation (FIG. 8H).

Conclusions

The main findings of this pilot study include:
ALM201 0.3 mg/kg treatment slightly reduced retinal inflammation clinically and histologically.
ALM201 3 mg/kg treatment significantly reduced retinal inflammation clinically and histologically.
Dexamethasone 0.5 mg/kg alone or Dexamethasone+ALM201 treatment significantly reduced retinal inflammation.
Low dose of ALM201 (0.3 mg/kg) treatment was able to prevent the progression or reduce inflammation in 20% of mice, and decrease the level of EAU progression in 80% of mice.
High dose of ALM201 (3 mg/kg) treatment was able to prevent the progression of inflammation in 30% of mice and reduce pre-existing inflammation in 30% mice.
Dexamethasone (0.5 mg/kg) treatment reduced pre-existing retinal inflammation in 30% of mice and prevented the progression of EAU in 40% of mice.
The combined therapy of Dexamethasone (0.5 mg/kg) and ALM201 (0.3 mg/kg) treatment reduced pre-existing retinal inflammation in 60% of mice and prevented the progression of EAU in 20% of mice.

Our results suggest that ALM201 dose-dependently suppressed retinal inflammation in the mouse model of EAU. The combination of Dexamethasone and ALM201 appears to have stronger immune suppressive effects than Dexamethasone or ALM201 alone. Potential mechanism of ALM201 may involve (1) preventing/reducing leukocyte trafficking from circulation into the inflamed retina; (2) modulating immune cell function and activation.

### Example 3—Distribution of ALM201 in the Eye Following Topical Administration and its Effect on Inflammation

Matrix-assisted laser desorption/ionization (MALDI) mass spectrometry imaging (MSI) was used to map the distribution of ALM201 in the eye.

Methods

On day 0, sutures where placed into the cornea of rats to induce corneal neovascularisation and inflammation. The eyes were treated for 3 days or 6 days with 16 μl of ALM201 (100 nM and 100 μM) or PBS (vehicle control).

Eyes were enucleated on the 3^{rd} or 6^{th} day one hour after treatment and frozen in gelatine.

Cross sections (10 μm) were taken at the centre of the eye for MSI, adjacent sections were stained with haematoxylin and eosin (H&E).

All MALDI-MS imaging experiments were performed using either MALDI-TOF/TOF (Ultraflextreme, Bruker Daltonics) or MALDI-FT-ICR (Solarix XR 12T, Bruker Daltonics) mass spectrometer in positive ion mode using a Smartbeam II 2 kHz laser. The laser spot size was selected to yield intermediate and sharp levels of focus (˜40 μm and 10 μm laser spot diameters) for low and high spatial resolution analyses. MS imaging data were visualized using FlexImaging (Bruker Daltonics), version 4.1. CHCA (5 mg/ml dissolved in 50% ACN containing 0.2% TFA) was used as MALDI matrix and applied using an automatic sprayer (TM-Sprayer, HTX Technologies).

Results

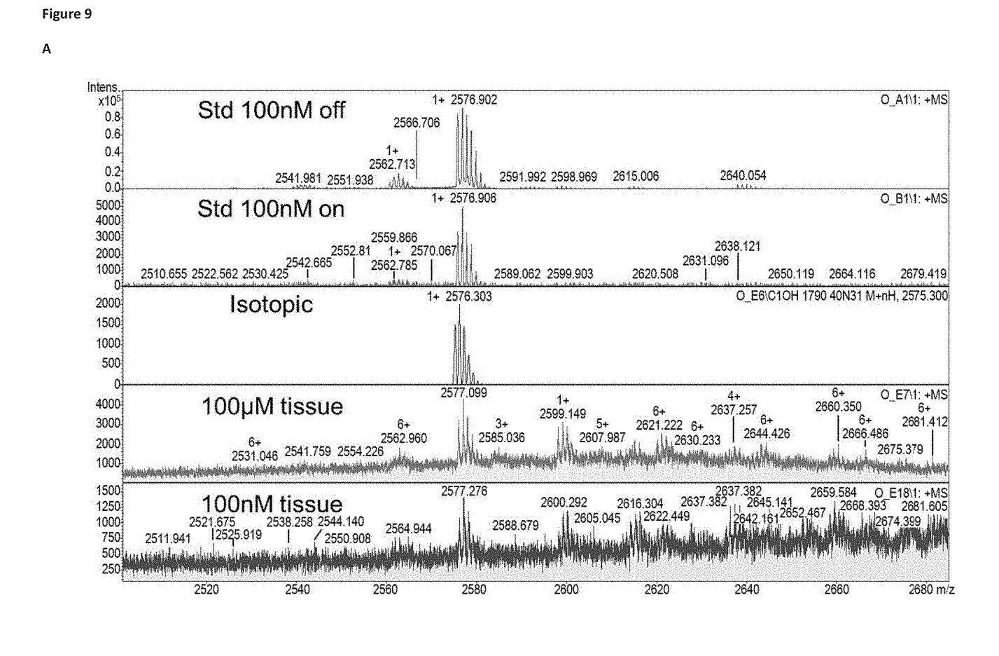

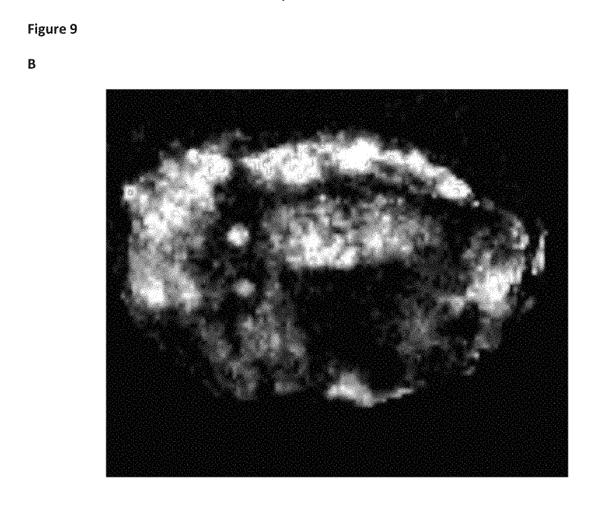

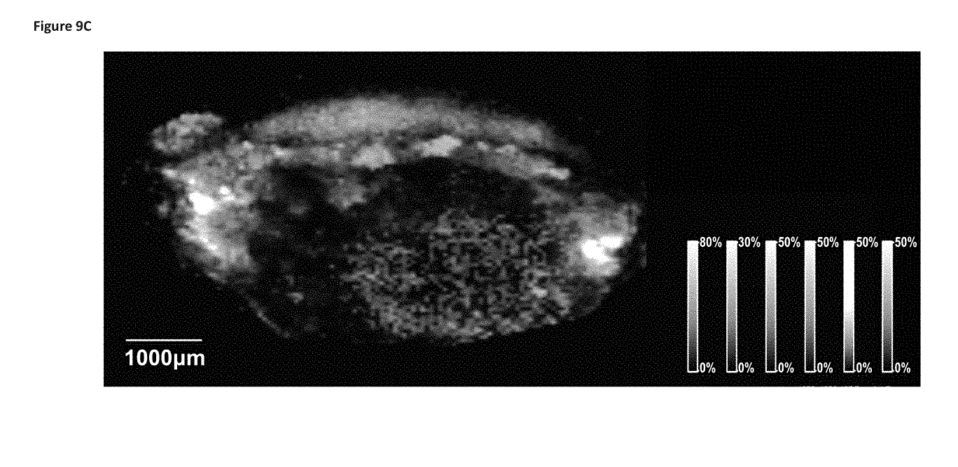

The MALDI-MS imaging results are shown in FIG. 9.

FIG. 9A shows FTICR Mass spectrum of ALM201 peptide at m/z 2576.303±0.005 Da. Results are shown from corneal tissue topically treated with 100 μM (penultimate trace) and 100 nM peptide (bottom trace). The observed peptide monoisotopic mass was in agreement with the theoretical distribution (mass accuracy was <5 ppm at 350K mass resolution power).

FIG. 9 B shows MALDI-FTICR-MSI heat maps distribution of ALM201 at m/z 2576.303±0.005 Da and FIG. 9C shows heat maps of: Endogenous metabolites at m/z 1028.135±0.025 Da mostly distributed in the cornea, 1444.584±0.025 Da mostly distributed in the lens, 782.5799±0.025 Da mostly distributed in the aqueous humour and vitreous humour, 780.5451±0.025 Da distributed in the muscle, 835.5891±0.025 Da and ALM201 distributed throughout the eye 2576.303±0.025 Da. These data demonstrate ALM201 had penetrated all layers of the eye following topical treatment.

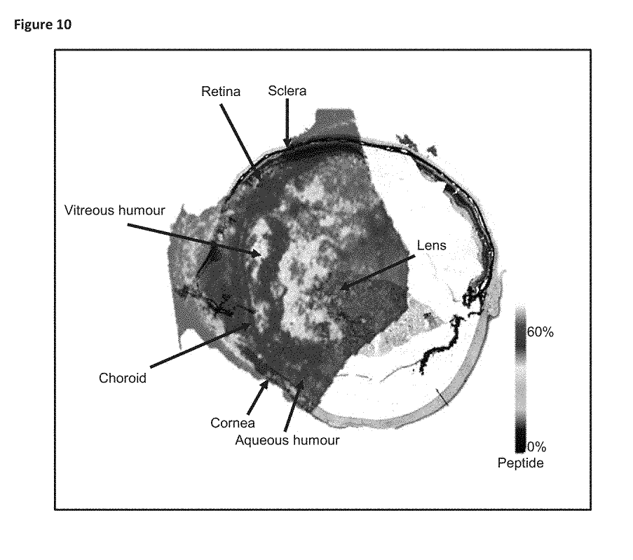

FIG. 10 shows a superimposition of H&E stained rat eye section and MALDI-MSI image at 2576.3±0.1 Da. A normal eye was treated daily with 100 μM of ALM201 for 3 days and then enucleated 15 minutes after the last treatment. As indicated by the heat map, the image shows that the majority of the peptide co-localises with the vitreous humour and possibly the lens. The peptide is also co-localised is the cornea, sclera, choroid and retina.

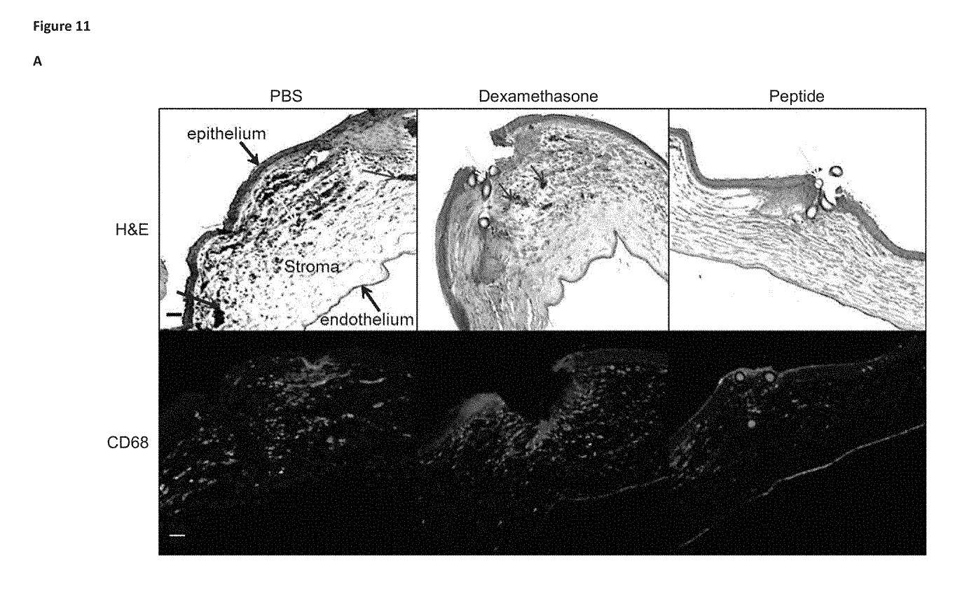

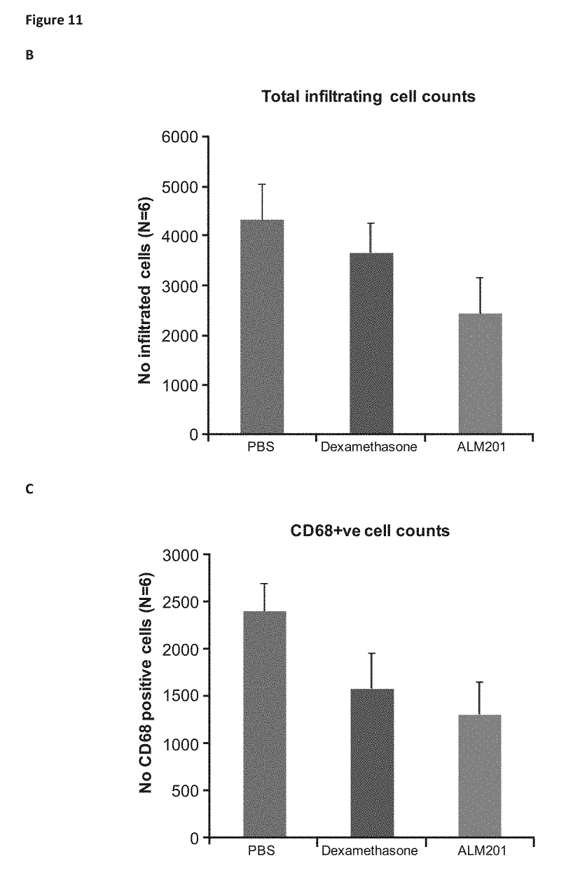

FIG. 11 shows the results of the histological analysis. Eyes from rats treated with ALM201 showed decreased neovascularisation compared to controls (FIG. 11A, white arrows indicate sutures and black arrows indicate blood vessels).

In addition, ALM201-treated animals showed reduced total cell infiltrates (FIG. 110B) and CD68+ cell infiltrates (FIG. 110) compared to both PBS-treated controls and also to dexamethasone-treated animals. CD68+ is a marker of macrophage and monocytes, and a reduction in the CD68+ cell infiltrate indicates reduced inflammation in these samples.

Conclusions

These data demonstrate that ALM201 administered topically to the cornea penetrates and distributes rapidly to all layers of the eye. Therefore ALM201 can provide effective treatment of anterior ocular diseases and posterior diseases. H&E and CD68+ staining shows topically administered ALM201 inhibits neovascularisation, total cell infiltration and inflammatory cell infiltrates,

### Example 4—Use of ALM201 in Preventing Neovascularisation and Inflammation in the Injured Cornea

ALM201 Concentration Titration

Method

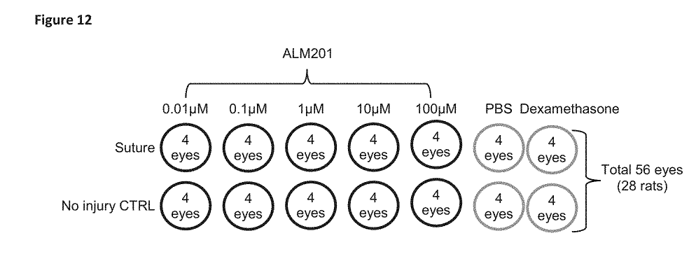

Each rat was anesthetised and two 10-0 sutures were placed intrastromally into the temporal cornea of the left eye, 1.5 mm from the limbus. The sutures were left in place for the entirety of the experiment. The right eye was kept as a no suture control but given the same treatment as the left eye. Treatment began approx. 24 hours after suture placement and continued once a day for 6 days. Rats were treated according to the treatment scheme shown in FIG. 12

Approximately 24 hours after the last treatment the eyes were imaged and clinically scored. The sutured eyes were scored based on vessel density, vessels distance to suture and inflammation. Vessel density was scored out of three, 0=no density, 1=mild, 2=moderate, 3=high. Vessels distance to suture was scored out of four, 0=no reach, 1=small distance, 2=moderate distance, 3=¾ of the way, 4=reached sutures and inflammation was scored out of three, 0=none, 1=minimal, 2=moderate, 3=severe. After clinical scoring the eyes were enucleated, tissue processed, wax embedded and cut into 5 μm sections while taking care to find the sutured area. Sections were stained with haematoxylin and eosin (H&E) to confirm suture location in each eye and adjacent slides were then used for immunohistochemistry to identify CD44+ cells and FKBPL expression as well NFκB and p-IκBα.

Results

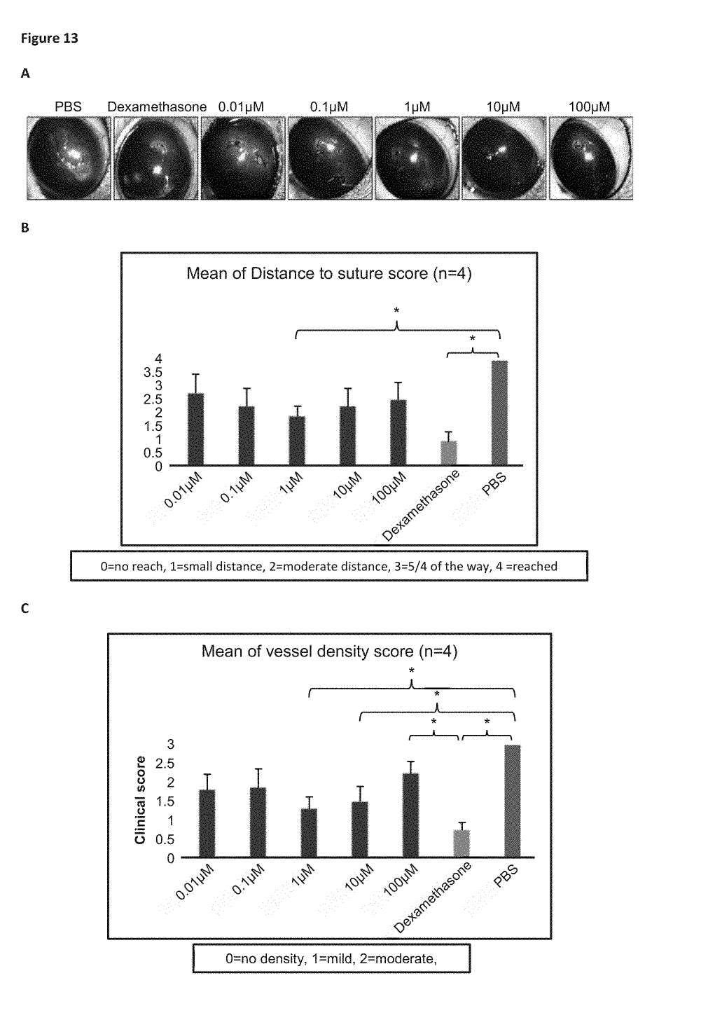

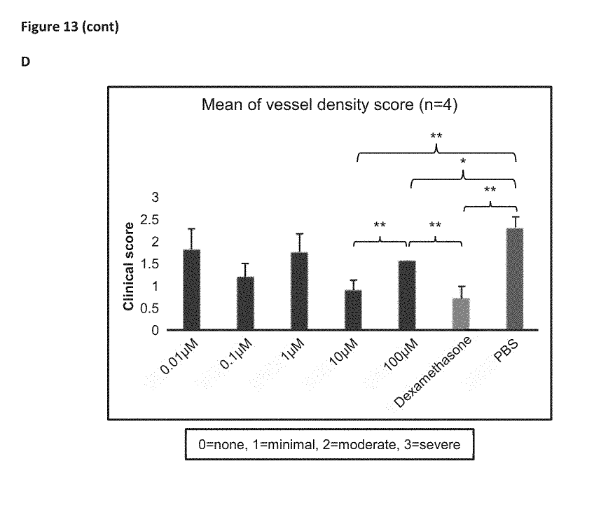

On day 7 after suture images were taken of each rat (FIG. 13A) and the corneal injuries were clinically scored. Results of clinical scoring showed that the vessel distance to sutures was significantly different in treatment groups 1 μM ALM201 and dexamethasone when compared to the PBS vehicle control (FIG. 13B).

Clinical scoring of vessel density also showed that there was a significant decrease in vessel density in ALM201 1 μM, 10 μM, 100 μM treated groups and dexamethasone when compared to PBS control group (FIG. 13C).

Inflammation scoring showed that there is a significant difference between dexamethasone, ALM201 10 μM and 100 μM in comparison to PBS treated group. There is also a significant difference in scores between 100 μM ALM201 and dexamethasone in both inflammation and vessel density scoring.

According to these results 1 μM and 10 μM were the most effective concentrations of ALM201 in preventing angiogenesis and inflammation.

Immunohistochemistry and H&E

Sections (5 μm) were stained with haematoxylin and eosin (H&E) to confirm suture location in each eye. Adjacent slides were then used for immunohistochemistry to identify, CD44+ cells and FKBPL expression.

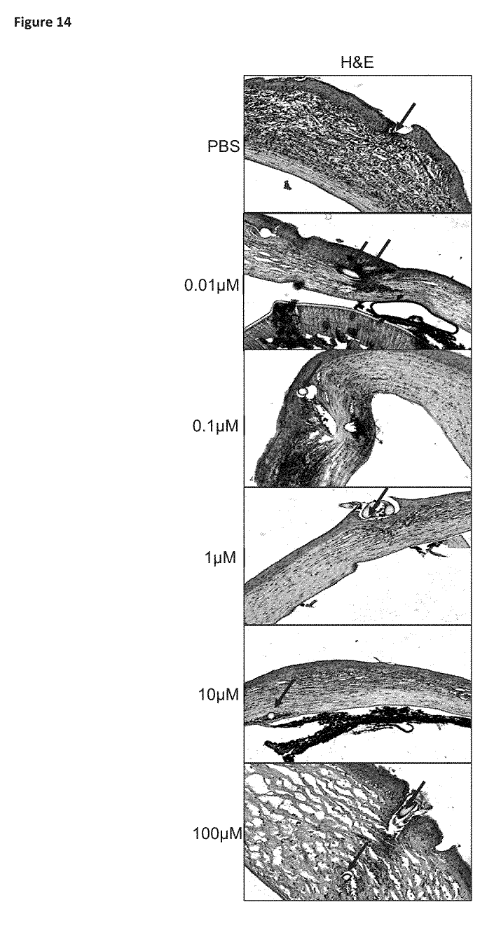

H&E staining (FIG. 14) showed that cell infiltrate, corneal swelling and blood vessels decreased as the ALM201 concentration was increased from 0.01 μM to 10 μM. However, at 100 μM ALM201 more corneal swelling was observed.

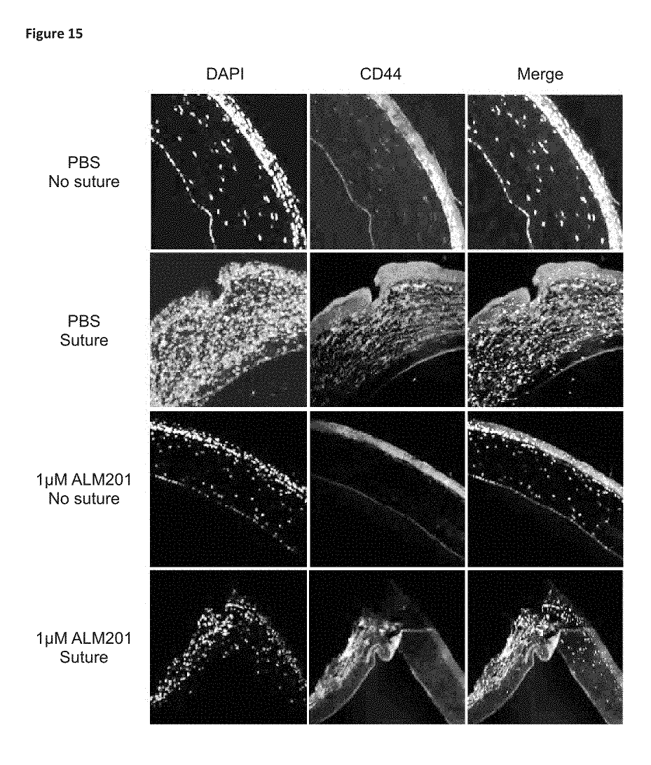

Immunohistochemistry staining for CD44 (FIG. 15) showed that CD44 is endogenously expressed in the corneal epithelium, stromal keratocytes and the corneal endothelium in a no suture cornea. In the PBS treated, sutured cornea CD44 expression was increased, most likely due to the increase in CD44+inflammatory cells in the stroma. Sutured corneas treated with 1 μM ALM201 showed a decrease in CD44 expression when compared to sutured PBS control. Therefore, it appears that 1 μM ALM201 is preventing CD44+inflammatory cells from entering the cornea.

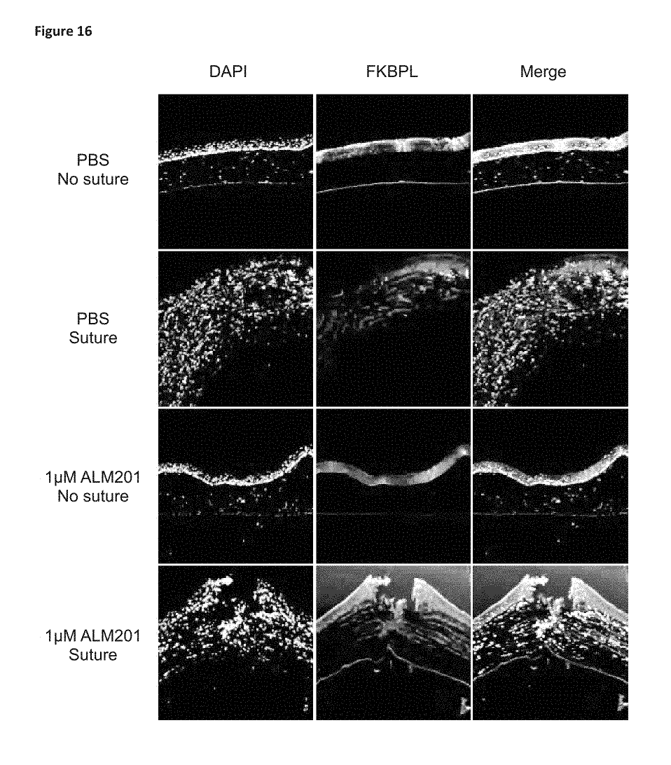

ALM201 is a derivative of the naturally occurring FKBPL protein. Immunohistochemistry of FKBPL (FIG. 16) showed that FKBPL is endogenously expressed in the corneal epithelium and endothelium in a normal cornea. In a sutured PBS treated cornea FKBPL is also expressed in the stroma. Interestingly, in sutured 1 μM ALM201 treated corneas FKBPL expression in the corneal epithelium and stroma increased in comparison with PBS treated sutured corneas and normal corneas (FIG. 16).

NFκB Pathway

NFκB and phosphorylated-IκBα antibodies were also used in this experiment to see if ALM201 affected the NFκB pathway. Nuclear factor kappa-light-chain-enhancer of B cells or NFκB is a transcription factor which is involved in several processes including inflammation, B cell development, apoptosis, immune regulation and cell proliferation. There are 5 different NFκB heterodimers in the NFκB family, depending on which heterodimer is activated they can activate or repress the transcription of the above process. The NFκB pathway is a very complex pathway which is activated by many different ligands such as TNFα, growth factors and IL-1b and acts on many different genes, including CD44. Anti-NFκB and phosphorylated-IκBα (p-IκBα) were used to discover whether ALM201 has an effect on this pathway to gain further insight into its mode of action.

In an unstimulated cell IκBα is bound to NFκB in the cytoplasm inhibiting NFκB nuclear localisation. When the NFκB pathway is activated IκBα is phosphorylated causing it to release NFκB unmasking its nuclear localisation signal. NFκB is then free to move into the nucleus where it functions as a transcription factor.

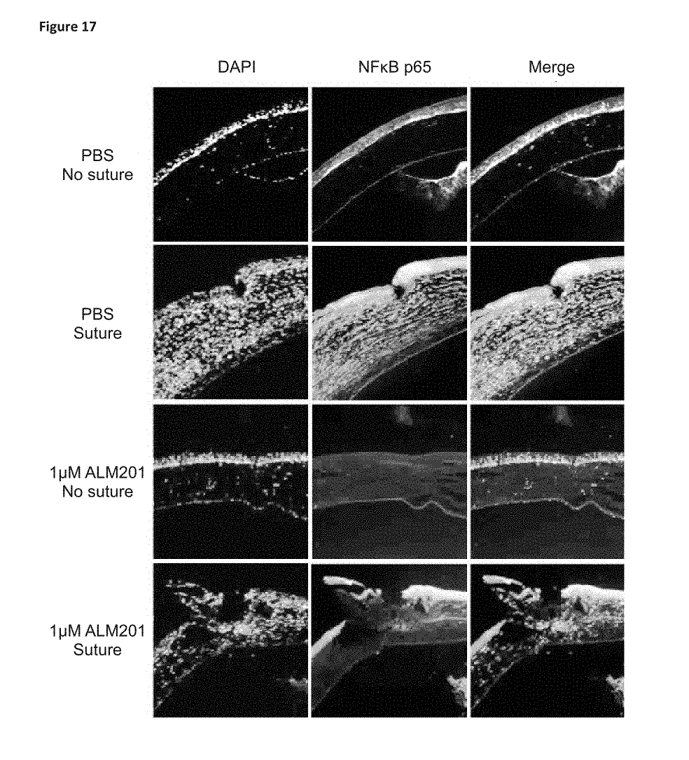

Immunohistochemical analysis of free NFκB expression (FIG. 17) showed that, in unsutured corneas, NFκB is endogenously expressed in corneal epithelium, endothelium and stromal keratocytes. Treatment with PBS or 1 μM ALM201 had no effect upon this pattern of NFκB expression in unsutured corneas. However, in sutured corneas treated with PBS, NFκB expression is increased when compared to the no suture controls. In contrast, sutured corneas treated with 1 μM ALM201 showed a reduction in NFκB expression in the stroma compared to PBS control sutured corneas.

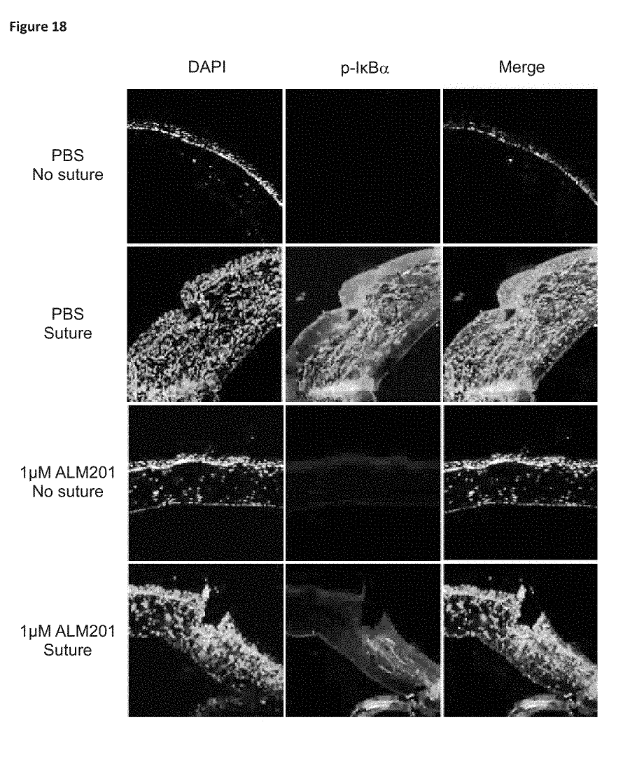

To confirm the observations of free NFκB expression, p-IκBα expression in the cornea was also immunohistochemically determined. There was no expression of p-IκBα in both PBS and 1 μM ALM201 treated unsutured corneas. In PBS treated sutured corneas there was an increase p-IκBα expression that was reduced by treatment with 1 μM ALM201 (FIG. 18).

From these results, ALM201 may be directly affecting the NFκB pathway or there may be a difference in expression due to the increased presence of inflammatory cells in the PBS treated and sutured corneas compared to the ALM201 treated corneas.

TABLE 3
FKBP-L peptides

| | SEQ |
|---|---|
| | ID |
| SEQUENCE | NO: |
| METPPVNTIGEKDTSQPQQEWEKNLRENLDSVIQIRQQPRDPPTETLELEVSPDPASQILEHTQGAEKLV | 1 |
| AELEGDSHKSHGSTSQMPEALQASDLWYCPDGSFVKKIVIRGHGLDKPKLGSCCRVLALGFPFGSGPPEG | |
| WTELTMGVGPWREETWGELIEKCLESMCQGEEAELQLPGHSGPPVRLTLASFTQGRDSWELETSEKEALA | |
| REERARGTELFRAGNPEGAARCYGRALRLLLTLPPPGPPERTVLHANLAACQLLLGQPQLAAQSCDRVLE | |
| REPGHLKALYRRGVAQAALGNLEKATADLKKVLAIDPKNRAAQEELGKVVIQGKNQDAGLAQGLRKMFG | |
| METPPVNTIGEKDTSQPQQEWEKNLRENLDSVIQIRQQPRDPPTETLELEVSPDPASQILEHTQGAEKLV | 2 |
| AELEGDSHKSHGSTSQMPEALQASDLWYCPDGSFVKKIVIRGHGLDKPKLGSCCRVLALGFPFGSGPPEG | |
| WTELTMGVGPWREETWGELIEKCLESMCQGEEAELQLPGHTGPPVGLTLASFTQGRDSWELETSEKEALA | |
| REERARGTELFRAGNPEGAARCYGRALRLLLTLPPPGPPERTVLHANLAACQLLLGQPQLAAQSCDRVLE | |
| REPGHLKALYRRGVAQAALGNLEKATADLKKVLAIDPKNRAAQEELGKVVIQGKNQDAGLAQGLRKMFG | |
| IRQQPRDPPTETLELEVSPDPAS (referred to herein as ALM201) | 3 |
| QIRQQPRDPPTETLELEVSPDPAS | 4 |
| METPPVNTIGEKDTSQPQQEWEKNLRENLDSVIQIRQQPRDPPTETLELEVSPDPASQILEHTQGAEKLV | 5 |
| AELEGDSHKSHGSTSQMPEALQASDLWYCPDGSFVKKIVIRGHGLDKPKLGSCCRVLALGFPFGSGPPEG | |
| WTELTMGVGPWREETWGELIEKCLESMCQGEEAELQLPGHTGPPVGLTLASFTQGRDSW | |
| METPPVNTIGEKDTSQPQQEWEKNLRENLDSVIQIRQQPRDPPTETLELEVSPDPASQILEHTQGAEKLV | 6 |
| AELEGDSHKSHGSTSQMPEALQASDLWYCPDGSFVKKIVIRGHGLDKPKLGSCCRVLALGFPFGSGPPEG | |
| WTELTMGVGP | |
| METPPVNTIGEKDTSQPQQEWEKNLRENLDSVIQIRQQPRDPPTETLELEVSPDPASQILEHTQGAEKLV | 7 |
| AELEGDSHKSHGSTS | |
| METPPVNTIGEKDTSQPQQEWEKNLRENLDSVIQIRQQPRDPPTETLELEVSPDPAS | 8 |
| METPPVNTIGEKDTSQPQQEWEKNLRENLDSVIQIRQQPRDPPTETL | 9 |
| QQPRDPPTETLELEVSPD | 10 |
| QIRQQPRDPPTETLELEVSPD | 11 |
| QIRQQPRDPPTETLELEV | 12 |
| QIRQQPRDPPTETLE | 13 |
| QIRQQPRDPPTE | 14 |
| QQPRDPPTETLELEVSPDPAS | 15 |
| RDPPTETLELEVSPDPAS | 16 |
| PTETLELEVSPDPAS | 17 |
| TLELEVSPDPAS | 18 |
| RQQPRDPPTETLELEVSPD | 19 |
| RQQPRDPPTETLELEVSP | 20 |
| RQQPRDPPTETLELEVS | 21 |
| PRDPPTETLELEVSPD | 22 |
| RDPPTETLELEVSPD | 23 |

1. FKBP-L polypeptide or a biologically active peptide fragment thereof for use in the treatment or prevention of corneal neovascularisation.

2. FKBP-L polypeptide or a biologically active peptide fragment thereof for use according to claim 1, for the prevention or treatment of corneal neovascularisation following corneal graft surgery

3. FKBP-L polypeptide or a biologically active peptide fragment thereof for use in the treatment or prevention of an inflammatory disorder of the eye.

4. FKBP-L polypeptide or a biologically active peptide fragment thereof for use according to claim 3, wherein said inflammatory disorder of the eye is uveitis.

5. FKBP-L polypeptide or a biologically active peptide fragment thereof for use according to claim 3, wherein said inflammatory disorder of the eye is dry eye syndrome.

6. FKBP-L polypeptide or a biologically active peptide fragment thereof for use according to claim 3, wherein said inflammatory disorder of the eye is blepharokeratoconjunctivitis.

7. FKBP-L polypeptide or a biologically active peptide fragment thereof for use according to any one of the preceding claims wherein said FKBP-L polypeptide or biologically active peptide fragment thereof is to be administered topically to the eye.

8. FKBP-L polypeptide or a biologically active peptide fragment thereof for use according to any one of claims 1 to 6 wherein said FKBP-L polypeptide or biologically active peptide fragment thereof is to be administered by subconjunctival injection, intrastromal injection or intraocular injection.

9. FKBP-L polypeptide or a biologically active peptide fragment thereof for use according to any one of claims 1 to 8, wherein said biologically active peptide fragment comprises the amino acid sequence IRQQPRDPPTETLELEVSPDPAS (SEQ ID NO:3), or a sequence at least 90% identical thereto.

10. FKBP-L polypeptide or a biologically active peptide fragment thereof for use according to any one of claims 1 to 8 wherein said FKBP-L polypeptide comprises the amino acid sequence shown as SEQ ID NO:1 or SEQ ID NO:2, or a sequence at least 90% identical thereto.

11. FKBP-L polypeptide or a biologically active peptide fragment thereof for use according to any one of claims 1 to 8 wherein said biologically active peptide fragment comprises the amino acid sequence shown as any one of SEQ ID Nos 4 to 23, or a sequence at least 90% identical thereto.

12. A method of treating or preventing corneal neovascularisation in a mammalian subject, comprising administering to a subject in need thereof a therapeutically effective amount of an FKBP-L polypeptide or a biologically active peptide fragment thereof.

13. The method of claim 12, wherein the subject to be treated has undergone corneal graft surgery.

14. A method of treating or preventing an inflammatory disorder of the eye in a mammalian subject, comprising administering to a subject in need thereof a therapeutically effective amount of an FKBP-L polypeptide or a biologically active peptide fragment thereof.

15. The method of claim 14 wherein the inflammatory disorder of the eye is uveitis.

16. The method of claim 14 wherein the inflammatory disorder of the eye is dry eye syndrome.

17. The method of claim 14 wherein the inflammatory disorder of the eye is blepharokeratoconjunctivitis.

18. The method of any one of claims 12 to 17 wherein the said FKBP-L polypeptide or biologically active peptide fragment thereof is administered topically to the eye.

19. The method of any one of claims 12 to 17 wherein the said FKBP-L polypeptide or biologically active peptide fragment thereof is administered by subconjunctival injection, intrastromal injection or intraocular injection.

20. The method of any one of claims 12 to 19 wherein said biologically active peptide fragment comprises the amino acid sequence IRQQPRDPPTETLELEVSPDPAS (SEQ ID NO:3), or a sequence at least 90% identical thereto.

21. The method of any one of claims 12 to 19 wherein said FKBP-L polypeptide comprises the amino acid sequence shown as SEQ ID NO:1 or SEQ ID NO:2, or a sequence at least 90% identical thereto.

22. The method of any one of claims 12 to 19 wherein said biologically active peptide fragment comprises the amino acid sequence shown as any one of SEQ ID Nos 4 to 23, or a sequence at least 90% identical thereto.

23. The method of any one of claims 12 to 22 wherein the subject to be treated is a human.


SEQUENCE LISTING (23 sequences)

SEQ ID NO: 1 - protein, 349 aa, Homo Sapiens
METPPVNTIGEKDTSQPQQEWEKNLRENLDSVIQIRQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGPWREETWGELIEKCLESMCQGEEAELQLPGHSGPPVRLTLASFTQGRDSWELETSEKEALAREERARGTELFRAGNPEGAARCYGRALRLLLTLPPPGPPERTVLHANLAACQLLLGQPQLAAQSCDRVLEREPGHLKALYRRGVAQAALGNLEKATADLKKVLAIDPKNRAAQEELGKVVIQGKNQDAGLAQGLRKMFG

SEQ ID NO: 2 - protein, 349 aa, Homo Sapiens
METPPVNTIGEKDTSQPQQEWEKNLRENLDSVIQIRQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGPWREETWGELIEKCLESMCQGEEAELQLPGHTGPPVGLTLASFTQGRDSWELETSEKEALAREERARGTELFRAGNPEGAARCYGRALRLLLTLPPPGPPERTVLHANLAACQLLLGQPQLAAQSCDRVLEREPGHLKALYRRGVAQAALGNLEKATADLKKVLAIDPKNRAAQEELGKVVIQGKNQDAGLAQGLRKMFG

SEQ ID NO: 3 - protein, 23 aa, Homo Sapiens
IRQQPRDPPTETLELEVSPDPAS

SEQ ID NO: 4 - protein, 24 aa, Homo Sapiens
QIRQQPRDPPTETLELEVSPDPAS

SEQ ID NO: 5 - protein, 199 aa, Homo Sapiens
METPPVNTIGEKDTSQPQQEWEKNLRENLDSVIQIRQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGPWREETWGELIEKCLESMCQGEEAELQLPGHTGPPVGLTLASFTQGRDSW

SEQ ID NO: 6 - protein, 150 aa, Homo Sapiens
METPPVNTIGEKDTSQPQQEWEKNLRENLDSVIQIRQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGP

SEQ ID NO: 7 - protein, 85 aa, Homo Sapiens
METPPVNTIGEKDTSQPQQEWEKNLRENLDSVIQIRQQPRDPPTETLELEVSPDPASQILEHTQGAEKLVAELEGDSHKSHGSTS

SEQ ID NO: 8 - protein, 57 aa, Homo Sapiens
METPPVNTIGEKDTSQPQQEWEKNLRENLDSVIQIRQQPRDPPTETLELEVSPDPAS

SEQ ID NO: 9 - protein, 47 aa, Homo Sapiens
METPPVNTIGEKDTSQPQQEWEKNLRENLDSVIQIRQQPRDPPTETL

SEQ ID NO: 10 - protein, 18 aa, Homo Sapiens
QQPRDPPTETLELEVSPD

SEQ ID NO: 11 - protein, 21 aa, Homo Sapiens
QIRQQPRDPPTETLELEVSPD

SEQ ID NO: 12 - protein, 18 aa, Homo Sapiens
QIRQQPRDPPTETLELEV

SEQ ID NO: 13 - protein, 15 aa, Homo Sapiens
QIRQQPRDPPTETLE

SEQ ID NO: 14 - protein, 12 aa, Homo Sapiens
QIRQQPRDPPTE

SEQ ID NO: 15 - protein, 21 aa, Homo Sapiens
QQPRDPPTETLELEVSPDPAS

SEQ ID NO: 16 - protein, 18 aa, Homo Sapiens
RDPPTETLELEVSPDPAS

SEQ ID NO: 17 - protein, 15 aa, Homo Sapiens
PTETLELEVSPDPAS

SEQ ID NO: 18 - protein, 12 aa, Homo Sapiens
TLELEVSPDPAS

SEQ ID NO: 19 - protein, 19 aa, Homo Sapiens
RQQPRDPPTETLELEVSPD

SEQ ID NO: 20 - protein, 18 aa, Homo Sapiens
RQQPRDPPTETLELEVSP

SEQ ID NO: 21 - protein, 17 aa, Homo Sapiens
RQQPRDPPTETLELEVS

SEQ ID NO: 22 - protein, 16 aa, Homo Sapiens
PRDPPTETLELEVSPD

SEQ ID NO: 23 - protein, 15 aa, Homo Sapiens
RDPPTETLELEVSPD